(19) United States
(12) Patent Application Publication
(10) Pub. No.: US 2026/0335182 A1
(43) Pub. Date: Oct. 29, 2026

(54) WEARABLE THERAPY DEVICES

(71) Applicant: Kineon, Inc., Atlanta, GA (US)

(72) Inventors: Christopher Lee Bohler, Peachtree City, GA (US); Yonatan Ali Rodriguez Arias, Mexico City (MX); Forrest Edward Smith, Atlanta, GA (US); Joseph Patrick Lee, Stoneham, MA (US); Aaron Robby Rogers, Eugene, OR (US); Mary Therese Lee, Stoneham, MA (US); Evan Peikon, Hurley, NY (US)

(21) Appl. No.: 19/658,115

(22) Filed: Apr. 24, 2026

Related U.S. Application Data
(60) Provisional application No. 63/793,853, filed on Apr. 24, 2025.

(51) Int. Cl. A61H 23/02; A61N 5/06

(52) U.S. Cl. CPC A61H 23/0254; A61N 5/0616; A61H 2201/10; A61H 2201/1215; A61H 2201/123; A61H 2201/165; A61H 2201/5005; A61H 2201/5084; A61N 2005/0626; A61N 2005/0645

(57) ABSTRACT

A vibration therapy apparatus may include a wearable band positionable on or around a body portion of a user. A vibration therapy apparatus may include a vibration module that can generate vibration and is adjustably connected to the wearable band. A vibration therapy apparatus may include a motion sensing module that can detect at least one of a movement or orientation of the wearable band and is attachable to the wearable band. A control unit may be attachable to the wearable band and is operative to receive real-time sensor data from the motion sensing module, generate one or more signals based on the real-time sensor data and one or more preset personalized vibration therapy settings stored in the control unit, and based on the one or more signals, cause the vibration module to dynamically adjust at least one vibration parameter associated with the vibrations.

### CROSS-REFERENCE TO RELATED APPLICATIONS

This application claims the priority benefit of 63/793,853, filed Apr. 24, 2025, the contents of which are herein incorporated by reference in their entirety.

### INCORPORATION BY REFERENCE

All publications and patent applications mentioned in this specification are herein incorporated by reference in their entirety, as if each individual publication or patent application was specifically and individually indicated to be incorporated by reference in its entirety.

### TECHNICAL FIELD

The present disclosure generally relates to wearable therapeutic devices and systems. More particularly, the disclosure relates to wearable apparatuses that provide personalized vibration‑based therapy for individual users. This disclosure also relates generally to systems and methods for delivering Photobiomodulation (PBM) to specific target tissues. The disclosed therapies are used for managing or treating inflammation, aches, pain, and other conditions.

### BACKGROUND

Vibration therapy has conventionally been used as a simple and non-invasive way to ease muscle pain, reduce fatigue, and stimulate local blood circulation. In some instances, vibration therapy may have been used to provide relaxation and movement necessary to maintain healthy muscles or the overall health of the user.

### SUMMARY

In some aspects, the techniques described herein relate to a vibration therapy apparatus including: a wearable band configured for placement on or around a body portion of a user; a vibration module adjustably connected to the wearable band and configured to generate vibrations; a motion sensing module attachable to the wearable band and configured to detect at least one of a movement or orientation of the wearable band; and a control unit attachable to the wearable band, the control unit being communicatively coupled to the vibration module and the motion sensing module, wherein the control unit is configured to: receive real-time sensor data from the motion sensing module, generate one or more signals based on the real-time sensor data and one or more preset personalized vibration therapy settings stored in the control unit, and based on the one or more signals, cause the vibration module to dynamically adjust at least one vibration parameter associated with the vibrations.

In some aspects, the techniques described herein relate to a vibration therapy system including: a vibration therapy apparatus including: a wearable band configured for placement on or around a body portion of a user; a vibration module adjustably connected to the wearable band and configured to generate vibrations; a motion sensing module attachable to the wearable band and configured to detect at least one of a movement or orientation of the wearable band; a control unit attachable to the wearable band, the control unit being communicatively coupled to the vibration module and the motion sensing module, wherein the control unit is configured to: receive real-time sensor data from the motion sensing module, generate one or more signals based on the real-time sensor data and one or more preset personalized vibration therapy settings stored in the control unit, and based on the one or more signals, cause the vibration module to dynamically adjust at least one vibration parameter associated with the vibrations; and an external computing device configured to communicate with the vibration therapy apparatus, the external computing device including a processor, a memory storing instructions of an application, and a storage unit configured to store data received from the vibration therapy apparatus, wherein, the processor is configured to execute the instructions to cause the external computing device to: display the one or more of: the at least one vibration parameter, the real-time sensor data, or the vibration therapy apparatus data using a user interface, and remotely cause the control unit to execute one or more of: adjust one or more vibration parameters in real-time, adjust the one or more preset personalized vibration therapy settings, or optimize the one or more vibration parameters based on one or both of: the vibration therapy apparatus data or the real-time sensor data by applying a machine learning method.

In some aspects, the techniques described herein relate to a device for delivering a photobiomodulation therapy, including: a base support; an attachment means for securing to a user; one or more module attachment sites for coupling to the base support; and one or more photobiomodulation modules adapted to reversibly attach to the one or more module attachment sites using the base support and through clothing material or garments.

In some aspects, the techniques described herein relate to a method of providing vibration therapy, the method configured to be performed by a control unit and including: receiving real-time sensor data from a motion sensing module and a tension sensing module or other sensors; generating one or more signals based on the received real-time sensor data and one or more preset personalized vibration therapy settings stored in the control unit; based on the one or more signals, causing: a vibration module to dynamically adjust at least one vibration parameter associated with vibrations generated by the vibration module, the tension sensing module to adjust a tension of a wearable band associated with the motion sensing module, the tension sensing module, and the vibration module, an orientation of the vibration module to align the vibrations along a desired direction to achieve the one or more preset personalized vibration therapy settings, and the vibration module to generate the vibrations consistent with the at least one vibration parameter and one or more present personalized vibration therapy settings.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIG. 1 illustrates a block diagram of a wearable vibration therapy apparatus, according to an embodiment of the present disclosure.

FIG. 2 illustrates a perspective view of a wearable vibration therapy apparatus with a detached vibration module, according to an embodiment of the present disclosure.

FIG. 3 illustrates an exploded view showing a detached vibration module, according to an embodiment of the present disclosure.

FIG. 4 illustrates a top view of the wearable vibration therapy apparatus, according to an embodiment of the present disclosure.

FIG. 5 illustrates a bottom view of the wearable vibration therapy apparatus, according to an embodiment of the present disclosure.

FIG. 6 depicts a flow diagram illustrating a method of operating the wearable vibration therapy apparatus, in accordance with an embodiment of the present disclosure.

FIG. 7 illustrates a block diagram of a system including the wearable vibration therapy apparatus, according to an embodiment of the present disclosure.

FIG. 8 illustrates a perspective view of a wearable photobiomodulation device with a detached magnetic ring, according to an embodiment of the present disclosure.

FIG. 9 shows a diagram of various vibration patterns that may be delivered through one or more embodiments of the present disclosure.

FIGS. 10A and 10B show a schematic of a vibration therapy apparatus comprising an inflatable element, according to an embodiment of the present disclosure.

FIGS. 10C and 10D show a schematic of a vibration therapy apparatus comprising an expandable vibration module, according to an embodiment of the present disclosure.

FIGS. 10E and 10F show a schematic of a vibration therapy apparatus comprising a spooling mechanism, according to an embodiment of the present disclosure.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the claimed subject matter. Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

Early vibration therapy devices were bulky and would apply a constant, single frequency vibration to the entire body of the user. To receive an effective vibration therapy, the user had to remain stationary during the entire session. Further, the user had the option to manually adjust the intensity of the vibrations by turning a dial or pressing a button. These vibration therapy devices may have offered temporary relief but lacked portability and limited movement of the user during a therapy session.

As electronics and battery technology improved, lighter vibration therapy devices such as vibration pads and handheld massagers were developed. These portable vibration therapy devices made it easier to target a specific body portion of the user. However, if the vibration therapy devices were not well-positioned, the targeted muscles or the body portion may not receive the full benefit of the vibration therapy. Handheld massagers may be used to overcome some of the positioning issues mentioned above but require continuous manual pressure, which is often inconvenient to the users.

More recently, wearable vibration therapy devices such as belts, sleeves, and pads have become popular. These wearable vibration therapy devices allow the users to freely move, sit, walk, or work while receiving localized or targeted vibration therapy. Many wearable vibration therapy devices deliver a few preset patterns and require the user to press buttons to change the intensity of vibrations or set a timer. However, the therapeutic effectiveness of the vibrations received from the wearable vibration therapy devices depends significantly on how tightly the wearable devices are worn around the body portion of the user. A loosely fit wearable vibration therapy device dissipates vibration energy and as a result, the user may not receive effective therapy using the vibration device. An overly tight device may cause discomfort or even restrict blood circulation. Further, these wearable vibration therapy devices offer little or no feedback on position or contact pressure to the body portion of the user. The users may have to continually reposition or readjust the vibration therapy settings or vibration parameters as they move, for example, when the user shifts posture or begins moving during a therapy session. Without onboard sensing, the wearable vibration therapy devices cannot compensate for loosening or posture changes. As a result, the consistency and quality of therapeutic effectiveness may vary widely.

Furthermore, many conventional wearable vibration therapy devices vibrate along a single axis or direction, which may not be sufficient to engage the targeted body portions or muscles uniformly or deeply. Some conventional wearable vibration therapy devices such as vibration belts combine vibration with heat and include several preset modes to increase the therapeutic effectiveness. However, these vibration belts still rely on the user to reposition the belt and readjust vibration intensity whenever the body posture changes. More recent wearable vibration therapy devices such as multi‑sensor fitness bands and posture‑feedback belts use sensors to capture motion or orientation data, enabling basic activity tracking or posture coaching. However, these devices generally do not control the vibration parameters in real-time.

In addition, many people suffer from conditions such as muscle aches and inflammation from acute (e.g., exercise related) and/or chronic causes. Drugs such as painkillers are used to manage aches, pain, inflammation, etc., but have side effects. The medical risks of drug therapy often increase with longer term use. Thus, drugs are often not a viable option in the long term. Exercise, yoga, etc. are often used, but they do not target specific pain or inflammation sites and are often ineffective.

In some embodiments, the disclosed vibration therapy apparatuses provide a technical solution to positional drift and misalignment during use. As the user moves, changes posture, or engages in activity, the relative orientation of the vibration module with respect to the target body portion may change. By continuously acquiring real-time motion and orientation data using the motion sensing module and tension data using the tension sensing module, the control unit can dynamically compensate for such misalignment by adjusting one or more vibration parameters and/or generating alerts or corrective adjustments. This automatic compensation reduces dependency on manual readjustment and maintains consistent therapeutic delivery despite body movement.

In some embodiments, the disclosed systems provide a technical solution for improving energy efficiency of wearable vibration therapy devices. By dynamically regulating vibration parameters based on real-time sensor feedback, the control unit avoids unnecessary over-vibration or excessive intensity when sufficient therapeutic coupling is detected. This adaptive control reduces power consumption of the vibration actuators and extends battery life, while still maintaining therapeutic effectiveness within the preset personalized therapy settings.

In some embodiments, the vibration therapy apparatus provides a technical solution for validating effective mechanical coupling between the vibration module and the body of a user. Tension measurements from the wearable band, in combination with motion data and vibration response characteristics, enable the control unit to infer whether vibrations are being properly transmitted to the target tissue. When insufficient coupling is detected, the system may adjust parameters, prompt the user, or pause therapy. This closed-loop validation improves consistency and safety of therapy delivery.

In some embodiments, the disclosed apparatus provides a technical solution for reducing user cognitive and operational burden. Instead of requiring repeated manual selection of vibration modes or intensity levels, the system automatically adapts therapy parameters in response to sensed conditions and personalized settings. This automation reduces user distraction and enables therapeutic sessions to occur passively while the user performs daily activities.

In some embodiments, the vibration therapy apparatus provides a technical solution for distinguishing between vibrations generated by the vibration module and movements caused by the body of the user. By applying frequency filtering, movement pattern recognition, and/or machine learning techniques to sensor data, the control unit differentiates therapy-induced vibrations from physiological motion. This differentiation enables accurate control decisions and prevents erroneous parameter adjustments.

In some embodiments, the disclosed vibration therapy apparatus provides a technical solution for anatomical variability among users. The modular vibration actuators may be repositioned, replaced, or oriented differently to accommodate different body portions, tissue depths, or therapeutic goals. This modularity allows a single platform to deliver customized therapy across a wide range of anatomical targets without redesigning the device.

In some embodiments, the disclosed apparatus provides a technical solution for reducing abrupt neuromuscular transitions at the end of therapy sessions. By gradually reducing vibration parameters using controlled ramp-down profiles, the system allows treated tissues to return toward baseline physiological states, improving comfort and reducing potential adverse sensations.

In some embodiments, a vibration therapy apparatus provides a technical benefit of being able to dynamically adjust one or more vibration parameters to enhance the therapeutic efficiency while not going beyond the preset personalized vibration therapy settings. For example, a vibration therapy apparatus may include a wearable band that is securely positionable on or around a body portion of a user. A vibration module can be adjustably connected to the wearable band and can generate vibrations. A motion sensing module may be attachable to, or incorporated into, the wearable band and can detect at least one of a movement or orientation of the wearable band. A tension sensing module may be attachable to, or incorporated into, the wearable band and can measure tension of the wearable band. The vibration therapy apparatus may also include a control unit attachable to, or incorporated into, the wearable band. The control unit can be communicatively coupled to the vibration module, the motion sensing module, and the tension sensing module to receive real-time sensor data and generate one or more signals based on received real-time sensor data. Further, one or more preset personalized vibration therapy settings may be stored in the control unit to dynamically adjust at least one vibration parameter associated with the vibration module.

In some embodiments, a vibration therapy apparatus provides a technical benefit of being able to independently adjust each vibration parameter based on real-time sensor data and/or the preset personalized vibration therapy settings to provide effective therapeutic sessions to the user. For example, the vibration module may receive one or more signals from the control unit to dynamically adjust at least one vibration parameter including a vibration intensity, a vibration frequency, or a vibration pattern.

In some embodiments, a vibration therapy apparatus provides a technical benefit of enabling targeted multi-axis vibration therapy to a user with desired therapeutic effectiveness. For example, the vibration module may include one or more modular vibration actuators, each structured to generate vibrations in at least one direction or axis. A position of each of the modular vibration actuators may be adjustable. A position of the modular vibration actuator(s) may be adjustable, and/or one or more modular vibration actuator may be replaceable to provide varying vibration therapy effectiveness in multiple directions or axes along the body portion of the user.

In some embodiments, a vibration therapy apparatus provides a technical benefit of providing vibration therapy along a length and/or a breadth of a body portion such as a muscle or group of muscles of the user by orthogonal placement relative to one another. For example, each modular vibration actuator of a pair may be positioned orthogonally relative to the other, or a pair of two pairs of modular vibration actuators may be positioned orthogonally relative to the other, to generate vibrations along orthogonal planes.

In some embodiments, a vibration therapy apparatus provides a technical benefit of enabling directed vibrations to a desired body portion of a user. For example, a vibration therapy apparatus may include a rotatable mechanical indexing mechanism for adjusting and/or maintaining a position of the one or more modular vibration actuators relative to the body part of the user.

In some embodiments, a modular vibration actuator includes a linear resonant actuator (LRA) or a pair of eccentric rotating mass (ERM) motors oriented at an angle relative to each other to generate vibrations along one or more different directions or axes.

In some embodiments, a vibration therapy apparatus provides a technical benefit of continuous monitoring of a movement and/or an orientation of the wearable band in real-time. For example, a motion sensing module may include an inertial measurement unit (IMU) having at least one accelerometer and at least one gyroscope to enable the continuous monitoring.

In some embodiments, a vibration therapy apparatus provides a technical benefit of being operable in a closed loop mode for providing effective therapeutic vibration therapy to the user. For example, the control unit of the vibration therapy apparatus may further include a processor and a memory storing instructions executable by the processor. Execution of the stored instructions by the processor cause storage of real-time sensor data, retrieval of historical usage data (e.g., including the one or more preset personalized vibration therapy settings, one or more vibration therapy parameters, etc.), and/or application of a machine learning model to: update one or more preset personalized vibration therapy settings associated with the user based on the real-time sensor data and/or the historical usage data; and dynamically control the at least one vibration parameter in real-time based on at least one of: the real-time sensor data and the updated one or more preset personalized vibration therapy settings.

In some embodiments, a vibration therapy apparatus provides a technical benefit of enabling consistent effective therapeutic vibration therapy by snuggly fitting the wearable band on or around the body portion of the user, while allowing the user to change position, walk, or perform activities. For example, the control unit may generate at least one of: an alert or automatically adjust tension of the wearable band based on real-time sensor data and/or the one or more preset personalized settings associated with the user.

In some embodiments, a vibration therapy apparatus provides a technical benefit of performing movement pattern recognition (e.g., using an artificial intelligence model) to recognize and differentiate between vibrations caused by the vibration therapy apparatus or by the movement of the body part of the user, thereby ensuring consistent effective therapeutic vibration therapy by snuggly fitting the wearable band on or around the body part of the user, while allowing the user to change position, or walk or do activities. For example, the control unit may distinguish between vibrations caused by the vibration module from movement of the body portion of the user by applying frequency filtering or movement pattern recognition.

In some embodiments, a vibration therapy apparatus provides a technical benefit of avoiding a sudden stop in a therapy session or at the conclusion of a therapy session, which could be jarring to the user and the treated area. For example, the control unit may gradually reduce at least one vibration parameter after a therapy session to allow the body portion of the user to settle or return to baseline or homeostasis.

In another aspect of the disclosure there is provided a method of providing vibration therapy using any of the vibration therapy apparatuses described herein. The method may include securing a wearable band of the vibration therapy apparatus on or around a body portion of a user. The vibration therapy apparatus may include a vibration module adjustably connected to the wearable band and structured to generate vibrations; a motion sensing module attachable to, or incorporated into, the wearable band for detecting at least one of a movement or orientation of the wearable band; a tension sensing module attachable to, or incorporated into, the wearable band for measuring tension of the wearable band; and a control unit attachable to, or incorporated into, the wearable band. The control unit may be communicatively coupled to the vibration module, the motion sensing module, and the tension sensing module to receive real-time sensor data and/or generate one or more signals based on received real-time sensor data and/or one or more preset personalized vibration therapy settings stored in the control unit to dynamically adjust at least one vibration parameter associated with the vibrations. The method may further include adjusting a tension of the wearable band and/or an orientation of the vibration module to align the vibrations along a desired direction and/or achieve one or more preset personalized vibration therapy settings and/or generate vibrations using the vibration therapy apparatus.

In some embodiments, the method may further include continuously detecting a movement and/or an orientation of the wearable band using the motion sensing module and monitoring the tension of the wearable band using the tension sensing module to generate real-time sensor data.

In some embodiments, the method may include dynamically adjusting one or more vibration parameters based on the real-time sensor data and/or the one or more preset personalized vibration therapy settings associated with a user and optionally automatically logging vibration therapy session data for personalization of one or more future therapy sessions.

In some embodiments, the method may further include automatically outputting an alert when deviations from a preset tension range and/or the one or more preset personalized vibration therapy settings are detected.

In some embodiments, the method may further include automatically adjusting a tension of the wearable band and/or adjusting the one or more vibration therapy parameters based on the real-time sensor data. In some embodiments, the method may employ one or more machine learning methods to analyze logged vibration therapy session data and update the one or more preset personalized vibration therapy settings and/or vibration therapy parameter(s) based on the real-time sensor data and/or the logged vibration therapy session data. The method may further include gradually reducing the at least one vibration parameter after a therapy session to allow the body portion of the user to settle, return to baseline, return to homeostasis, etc.

According to another aspect, the present disclosure includes a vibration therapy system having a vibration therapy apparatus. The vibration therapy system may include an external computing device for communicating with the vibration therapy apparatus. The external computing device includes a processor, one or more memory(ies) storing instructions of an application, and a storage unit to store data received from the vibration therapy apparatus. The processor, upon execution of the instructions, can cause the external computing device to: display the one or more vibration parameters and/or real-time data and/or stored sensor data using a user interface of the system; remotely adjust the one or more vibration parameters and/or the preset personalized vibration therapy settings in real-time using the control unit; and/or optimize the one or more vibration therapy parameters based on historical data and/or real-time feedback (e.g., through one or more sensors) by applying one or more machine learning (ML) methods or models.

One or more ML models may be implemented in a hierarchical approach with lightweight models for real-time adjustment of one or more therapy (e.g., vibration, PBM, etc.) parameters and complex models for long-term optimization. In some embodiments, one or more parameters may be optimized before or during or after a use session based on the profile of a user and/or stored historical use data. In some embodiments, parameter optimization may be performed through Bayesian Optimization (i.e., Gaussian Processes), where the relationship between therapy parameters and the desired outcome is modeled as a probability distribution. In some embodiments, parameter optimization may be performed through Contextual Multi-Armed Bandits (MAB). In some embodiments of MAB, each parameter is set as an arm to pull and the context is the baseline data of the user (e.g., sensor data, user inputs, etc.). In some embodiments, one or more parameters may be adjusted during use (e.g., in real time) using Long Short-Term Memory (LSTM) networks, Temporal Convolutional Networks (TCNs), Online Gradient Descent or other methods. Such methods may be used to interpret one or more data streams (e.g., sensor data, user feedback, use data, accelerometry data, etc.) of sequential data to detect if one or more parameters being used are sub-optimal. If sub-optimal, one or more parameters may be adjusted. Such methods may be used to detect drift of one or more data (e.g., sensor data, user feedback, use data, accelerometry data, etc.) from a starting point or from optimal levels. Online Gradient Descent-based techniques may use models that update their weights as new user feedback, sensor feedback, etc. is obtained.

The external computing device may monitor real-time sensor data and remotely control the one or more vibration parameters including vibration intensity, vibration frequency, and/or vibration patterns during one or more therapy sessions. The external computing device may store data associated with one or more of the therapy sessions to track user progress and adjust the one or more vibration parameters or the preset personalized vibration therapy settings for future therapy sessions based on historical patterns.

In some embodiments, one or more vibration therapy modules or systems may be used with one or more PBM devices or systems, for example, any of the systems described in U.S. Patent Application Ser. No. 19/298,879, filed August 13, 2025, which is herein incorporated by reference in its entirety. In some embodiments, one or more PBM modules/systems or components or treatment targets or methods, etc. described in U.S. Patent Application Ser. No. 19/298,879 may be incorporated in or used with one or more vibration therapy modules or systems described herein.

One or more vibration-based methods and elements may be used synergistically with one or more PBM based methods and elements. In some embodiments, each modality may target separate, or overlapping, biological pathways. In some embodiments, PBM may be delivered that is absorbed by cytochrome C oxidase in the mitochondria. In some embodiments, PBM may be delivered that performs one or more of: stimulating the electron transport chain, enhancing ATP production, modulating inflammatory pathways, and reducing cytokine activity. Once such healing pathways are targeted through PBM, other biological functions (e.g., tissue/cell movement, blood and other fluid circulation, and fluid exchange, etc.) may be used to complement the action of the PBM. Vibration-based modalities may be used for functions such as: mechanical stimulation to increase blood flow, enhancing lymphatic drainage, activating muscles reflexively, and modulating pain signals. Such combination or synergistic modalities may be used for one or more of: dilating vasculature through vibrations to drive oxygenated blood into the treated zone to enhance PBM efficacy, improving circulation through vibrations to increase anti-inflammatory activity of PBM (e.g., reduced cytokine activity, lowered localized swelling, etc.), increased lymphatic drainage through vibrations that enhances nitric oxide production through PBM, targeting specific muscles using a combination approach, modulating neurological pain using specific vibration frequencies (e.g., about 100 Hz and about 250 Hz) by targeting mechanoreceptors, enhancing neuronal-like cell growth and differentiation through specific vibration frequencies (e.g., around 40 Hz), etc.

One or more methods and device designs may be used to prevent vibrations from mechanically disrupting optical coupling. Examples of methods and devices to create a sufficiently stable tissue contact between one or more device portions and bodily region(s) while delivering a combination of PBM and vibrations including, but are not limited to:
1. Interleaved operation: In such methods, PBM and vibrations are delivered in alternating duty cycles (e.g., about 50 ms PBM → about 50 ms vibration). This may be used to avoid optical scatter from vibrating tissue while still delivering both modalities in rapid succession. In some embodiments, PBM may be delivered such that its cellular effects persist for more than a minute to allow this synergistic operation.
2. Zone-separated head: PBM light-emitting diodes (LEDs) or other elements may be placed on one or more devices such that they surround a central vibration element. The vibration may be delivered from the center while PBM may be delivered to the surrounding tissue.
3. Acoustic gel coupling layer: In some embodiments, a conformable gel pad between the transducer and tissue may be used that simultaneously acts as an optical window (transparent to red/near infrared (NIR) or other PBM wavelengths) and an acoustic coupler.

Conventional PBM therapies are limited by ease of use, efficacy, and/or portability. Further, such devices and methods cannot be easily adjusted and adapted for the specific needs of the user. Conventional PBM therapies cannot be easily applied to specific regions of the body such as the back of the abdomen, back of the neck, lower back, etc. For example, many women suffer from severe pain due to uterine cramping, endometriosis, and other conditions. Conventional PBM therapies cannot be placed flexibly and easily tailored to the needs of the individual user. The devices described herein solve the above technical problems with technical solutions. For example, the wearable devices described herein are designed to be positioned easily, effectively, and with stability at one or more target locations on the body. The devices may also be repositioned. The modular design of the devices disclosed herein may be used to deliver therapy at multiple body regions where the therapy parameter(s) may vary across the modules.

In some embodiments, a photobiomodulation device may be mounted onto standard clothing (shirt, pant, undergarment) by sandwiching the garment between the module which contains a fastening feature to a ring or other shaped object that contains a mating feature to the module. The nature of the mechanical connection between the two mating parts can take the form of a mechanical fit mechanism (male protrusion, pin, profile on one side and female mating part on the other) or the connection may be made via magnetic means with one part comprising or containing a magnetic material and the other comprising or containing a ferrous metal. In this embodiment, the photobiomodulation device may be mounted anywhere based on the extent of the clothing worn by the user.

In some embodiments, the systems disclosed herein provide a technical solution by integrating vibration therapy and photobiomodulation (PBM) within wearable and modular platforms. For example, PBM therapy is a non-invasive therapy where light of specific wavelengths is applied to tissues to induce specific biological effects. PBM therapy has been used to produce therapeutic effects such as reduction of inflammation and pain and promotion of tissue healing. The systems and devices described herein maintain consistent, comfortable stimulation; compensate for body movement and/or strap tension changes; and adapt to user‑specific vibration therapy preferences without frequent manual intervention. The coordinated or independent operation of vibration modules and PBM devices enables multimodal therapeutic delivery targeting inflammation, pain, and tissue repair. This integration allows therapies to be spatially and temporally tailored to specific body regions without increasing user complexity.

One or more of the devices and methods disclosed herein may be used individually or together for various uses. Examples of which include, but are not limited to:

Induce pain relief. The therapies disclosed herein may be used to generate or cause an analgesic effect. The therapies disclosed herein may be used to reduce pain perception. The therapies disclosed herein may be used to modulate the activity of peripheral nerves, potentially increasing the pain threshold. The therapies disclosed herein may be used to trigger the release of endorphins and other natural analgesic substances that reduce pain signals sent to the brain. Such mechanisms of actions can be used for easing pain due to endometriosis, adenomyosis, and other conditions.

Prompt cell regeneration and repair. PBM triggers cell repair and regeneration primarily through its interaction with the mitochondria, the energy-producing power houses of the cells. Through absorption and modulation of the enzyme cytochrome-c-oxidase (CCO), an increase in the production of adenosine triphosphate (ATP) occurs. With more ATP, cells perform their functions more efficiently, including cell repair and regeneration. This process also stimulates the release of growth factors which play a central role in tissue repair and regeneration.

Reduce Inflammation. The therapies disclosed herein may be used to inhibit pro inflammatory cytokines, which are molecules that promote inflammation. This may be used for reducing back pain since chronic inflammation can contribute to conditions such as sciatica, disc herniations, or general muscular strain. The reduction in inflammatory markers may also help to decrease pain and promote tissue repair. Such anti-inflammatory effects can be used for conditions such as endometriosis, chronic endometritis, etc.

Enhance blood flow and oxygenation. The therapies disclosed herein may be used to improve blood circulation by promoting vasodilation, or the widening of blood vessels. Enhanced circulation may be used to bring more oxygen and nutrients to an injured area and/or flush out toxins and waste products, thereby accelerating the tissue healing process. In patients with back pain, improved circulation may be used for faster recovery of strained muscles, tendons, and ligaments.

Promote post-surgical recovery. The therapies disclosed herein may be used to accelerate healing after abdominal and/or pelvic surgeries by reducing inflammation, pain, etc.

Improve Fertility. The therapies disclosed herein may be used to improve fertility, for example, by improving blood flow.

Induce muscle relaxation. The therapies disclosed herein may be used to relax muscles and reduce spasms. Such spasms may produce back pain and other pain conditions. Relaxation of muscles may be used to improve flexibility and range of motion, thereby providing relief from pain caused by muscle stiffness.

Stimulate a gut-brain connection. The therapies disclosed herein may be used to influence functioning of the gut by one or more of: influencing the gut microbiome (e.g., by increasing gut microbiome diversity), reducing inflammation in one or more gut regions, and/or modulating neurotransmitter production. This in turn may be used to influence brain functions such as improving mood and cognitive functions.

Stimulate collagen production. The therapies disclosed herein may be used to stimulate collagen synthesis, which in turn can be used for improving repair of tissues that may be contributing to pain.

Modulate the levels of molecules involved in the inflammation process. Examples of such molecules include, but are not limited to: (a) C-reactive protein (CRP): a protein that increases in response to inflammation; (b) cytokines (e.g., IL-6, TNF-α): small proteins released by cells that play a key role in regulating inflammation; (c) prostaglandins: lipid compounds that contribute to inflammation and pain; and (d) nitric oxide (NO): a signaling molecule that can indicate oxidative stress and inflammation.

Various vibration therapy devices and systems and photobiomodulation devices and systems will now be described through various embodiments. Even though only a few embodiments have been disclosed, modification of these embodiments and other embodiments may be utilized, without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

The present disclosure relates to a wearable vibration therapy apparatus for personalized and adaptive therapeutic applications. The wearable vibration therapy apparatus may include several interconnected components, each contributing to the effectiveness of the therapeutic vibrations. The detailed descriptions provided herein clarify the structure, operational methodology, and practical applications of the wearable vibration therapy apparatus, supported by reference to accompanying illustrative figures.

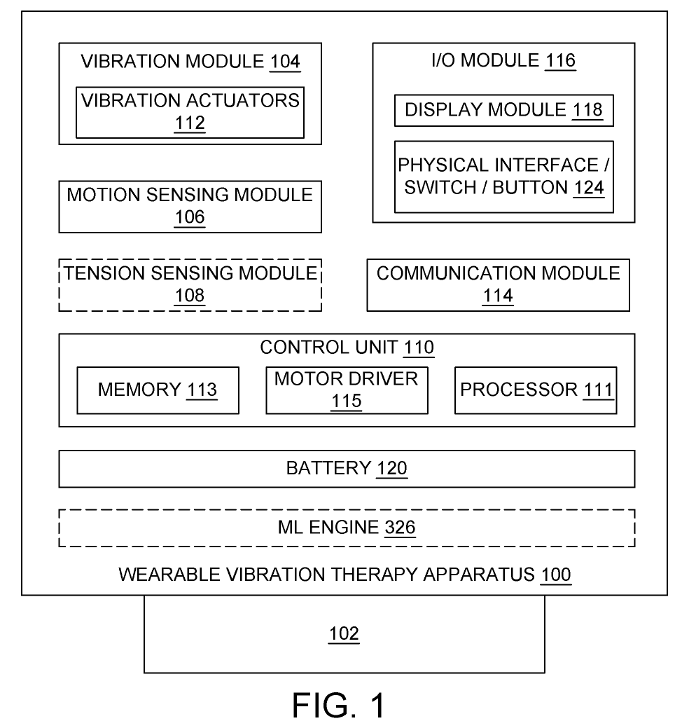

Referring to FIG. 1, a wearable vibration therapy apparatus 100 for providing dynamic and personalized vibration therapy to users is shown. The wearable vibration therapy apparatus 100 may include a wearable band 102 for secure placement on or around a body part of a user. The wearable vibration therapy apparatus 100 may also include a vibration module 104 for generating vibrations. The vibration module 104 may be adjustably connected to the wearable band 102. The vibration module 104 may include one or more modular vibration actuators 112 for generating vibrations in one or more directions or axes. The wearable vibration therapy apparatus 100 may further include a motion sensing module 106 attachable to, or incorporated into, the wearable band 102. The motion sensing module 106 may detect a movement and/or an orientation of the wearable band 102, which may occur when the user moves. The wearable vibration therapy apparatus 100 may also include a tension sensing module 108 attachable to, or incorporated into, the wearable band 102. The tension sensing module 108 is typically integrated into the wearable band 102 and can continuously monitor or measure tension of the wearable band 102.

The wearable vibration therapy apparatus 100 may further include a control unit 110 attachable to, or incorporated into, the wearable band 102 to control the various operations of the apparatus 100. The control unit 110 may be communicatively coupled to the vibration module 104, the motion sensing module 106, and/or the tension sensing module 108 using a communication module 114 to receive real-time sensor data. The real-time sensor data may include one or many inputs from any of these sensing modules, among other signals. The control unit 110 may generate signals based on one or more of: the received real-time sensor data, one or more preset personalized vibration therapy settings associated with the user, and/or vibration therapy parameters. The preset personalized vibration therapy settings associated with the user may be stored in the control unit to enable dynamic adjustment of one or more vibration parameters associated with the user. Any of the devices and methods described herein may be designed to prevent injury and/or discomfort to the user. Such injury and/or discomfort may be caused due to factors such as restricted blood flow, hypoxia, etc. In some embodiments, one or more preset personalized vibration therapy settings may be designed to prevent injury and/or discomfort to the user. In some embodiments, one or more preset personalized vibration therapy settings may be designed to avoid excessive restriction of blood flow to one or more bodily regions. In some embodiments, one or more preset personalized vibration therapy settings comprise one or more limits that prevent a user from setting incorrect or unsafe or inadequate vibration parameters.

In some embodiments, the one or more modules (such as a vibration module, a motion sensing module, a tension sensing module, etc.) may be physically and electrically coupled to a main module, such as the control unit and/or vibration module, using a variety of attachment mechanisms. In some embodiments, a module (such as a vibration module, a motion sensing module, a tension sensing module, etc.) may be integrated within a housing of the main module, such that the module and main module share a common enclosure. In this configuration, the module may be fixedly mounted to an internal structure of the main module to maintain a known spatial relationship, enabling accurate sensing of motion, orientation, or vibration. In some embodiments, a module (such as a vibration module, a motion sensing module, a tension sensing module, etc.) may be detachably mounted to the main module. For example, the module may be removably coupled using mechanical fastening features such as clips, snap-fit connectors, rails, or keyed interfaces that allow the module to be attached, removed, or repositioned relative to the main module. In some embodiments, the module (such as a vibration module, a motion sensing module, a tension sensing module, etc.) may be attached to the main module via magnetic coupling, allowing for repeatable alignment and user-friendly attachment while maintaining sufficient retention during movement of the user. Magnetic attachment may also facilitate modular interchangeability of sensor modules. In some embodiments, the module (such as a vibration module, a motion sensing module, a tension sensing module, etc.) may be mounted on or embedded within the wearable band and communicatively coupled to the main module using wired or wireless electrical connections. In such configurations, the positional relationship between the module and the main module is maintained through the wearable band, enabling coordinated sensing and control. In some embodiments, multiple modules may be attached to the main module at different locations or orientations, allowing data to be captured from different regions of the wearable apparatus. This arrangement enables the control unit to correlate data from multiple sources to improve accuracy of motion detection, tension measurement, and closed-loop control of vibration parameters. Each of the above attachment configurations allows the sensor modules to be operatively coupled to the main module while supporting modularity, repositioning, and adaptability to different body portions and therapeutic applications.

The wearable vibration therapy apparatus 100 may further include an input/output (I/O) module 116 to enable connection to external devices or for allowing users to interact with the apparatus 100 such as for controlling one or more vibration parameters of the wearable vibration therapy apparatus 100. In various embodiment, the I/O module 116 allows the users to interact with the wearable vibration therapy apparatus 100 using one or more physical buttons. Furthermore, the I/O module may include a display module 118 (also shown in FIG. 3), which allows the users to view the sensor data and/or other information related to the vibration or information related to the wearable vibration therapy apparatus 100. In some implementations, the display module 118 is a touch screen device allowing the users to view data related to the operations of the apparatus or any other information made available to the user via the display module 118. Additionally, or alternatively, the touch screen device 118 may enable a user to provide instructions to the apparatus 100 such as those for controlling the various parameters of the vibrations and/or to adjust a tightness or tension of the wearable band 102.

The wearable vibration therapy apparatus 100 may be powered using a battery 120 of sufficient capacity, which can be easily recharged, or in some implementations, can be replaced as needed. In some instances, the battery 120 may be housed in the same enclosure or housing as that of the control unit 110, which may also power the vibration module 104. In some other embodiments, the control unit 110 and the vibration module 104 are independently powered by batteries housed in their respective housings or enclosures. The battery 120 may be a rechargeable lithium-ion or lithium-polymer battery pack. In yet another embodiment, the battery 120 may be integrated into the wearable band 102 along with the control unit 110.

The wearable vibration therapy apparatus 100 may further include appropriate safety circuitry for voltage regulation and to provide stable power to the vibration module(s) 104 and the control unit 110 including various sensors. Further, the I/O module 116 may include one or more ports such as universal serial bus-C (USB-C) to allow for battery charging. In some embodiments, the wearable vibration therapy apparatus 100 may include appropriate hardware and software arrangements to support charging, including wireless charging of the battery 120. Further, the wearable vibration therapy apparatus 100 may include power management firmware that can monitor the battery level and optionally adjust the operation of the vibration module 104, for instance, by reducing intensity or alerting the user when the battery 120 is running low, thereby avoiding abrupt interruptions in therapy.

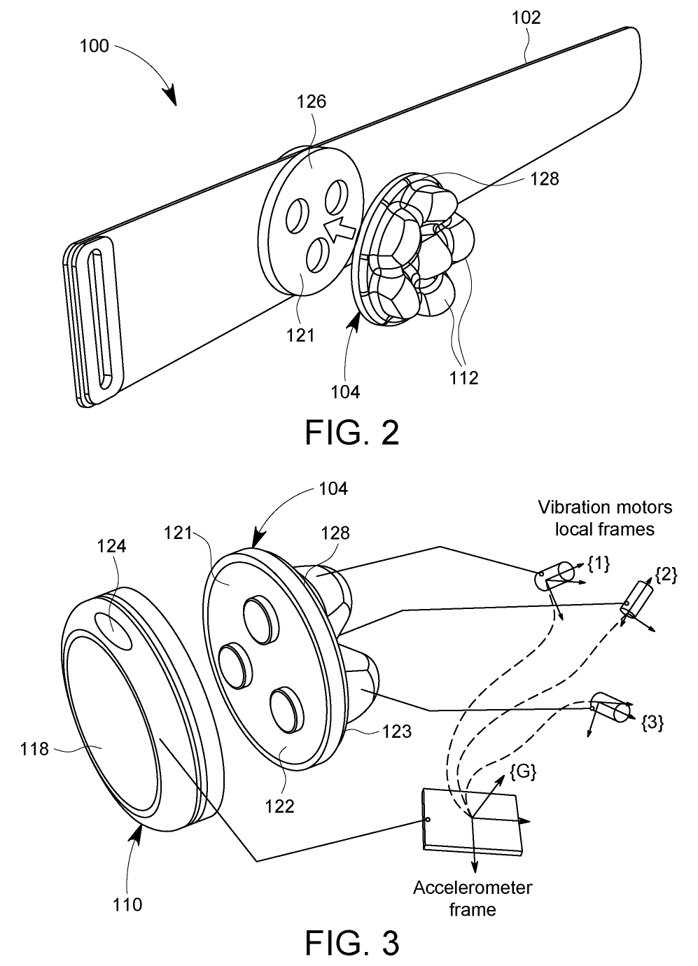

Referring now to FIG. 2, a wearable vibration therapy apparatus 100 is shown with a wearable band 102 and a detachable vibration module 104, according to an embodiment. In some embodiments, the vibration module 104 is modular, i.e., includes detachable or reconfigurable components that can be individually attached or removed from the vibration module 104.

In some embodiments, as shown in FIGS. 2-3, the vibration module 104 may have a base portion 122 having a top surface 121 opposite a bottom surface 123. The top surface 121 of the base portion 122 may detachably receive one or more of the vibration actuators 112, each configured to generate vibrations in one or more directions or axes using one or more vibration generating elements oriented to generate vibrations in one or more directions or axes. Once installed, each of the modular vibration actuators 112 on the top surface 121 of the base portion 122 can be manually adjusted or repositioned. Adjustments of the vibration actuators 112 may include manually removing, replacing, and/or installing a new vibration actuator 112, with higher or lower intensity settings, depending on therapy use cases or requirements. In some embodiments, one or more of the vibration actuators 112 are permanently attached to the base portion 122 or other apparatus 100 components.

In some embodiments, the base portion 122 may be circular in shape and may detachably receive the vibration actuators 112 on a top surface 121. However, alternative shapes, such as square or polygonal, may also be used, each structured to support the detachable mounting of one or more vibration actuators 112.

In some embodiments, one or more vibration actuators 112 are selected and arranged on the base portion 122 of the vibration module 104 to generate multi-axis vibrations for a therapy session. In some embodiments, a position of one or more vibration actuators 112 may be adjusted to focus vibrations in a specific direction. Further, the placement and configuration of the vibration actuators 112 may allow the application of multi-axis vibrations, for example during a therapy session. An angle and/or an orientation of one or more vibration actuators 112 may be adjusted individually or as a set to fine-tune the direction and/or type of therapeutic vibration delivered.

In some embodiments, the vibration actuator(s) 112 may include a linear resonant actuator (LRA) or two or more eccentric rotating mass (ERM) motors. Each actuator may be positioned on the base portion 122 at varying orientations. For example, one ERM motor may be aligned to vibrate in a horizontal plane, while another motor may be oriented to vibrate in a vertical plane relative to a body surface of the user. This configuration produces multi-axis, orthogonal vibrations that enhance therapeutic outcomes by stimulating both deep and surface-level muscle groups. In one implementation, the control unit 110 may drive each actuator or ERM motor either synchronously or with controlled phase differences, creating complex vibration patterns. These patterns can penetrate tissue more effectively and reach the intended muscles from various directions. This multi-axis approach enables the vibration actuators 112 to engage a greater number of muscle fibers and nerve endings, potentially improving blood circulation and providing more effective relief than one-dimensional vibration therapy.

In some embodiments, the wearable band 102 may include a mounting structure 126 integrated into the wearable band 102. In another implementation, the mounting structure 126 is detachably connected to the wearable band 102. The mounting structure 126 may attach the control unit 110 to the base portion 122 of the vibration module 104 through a rotatable mechanical indexing mechanism 128. In one implementation, the mounting structure 126 detachably attaches to the base portion 122 of the vibration module 104 using magnetic or other mechanical engagement means. Further, the mounting structure 126 may allow for direct electrical and communication connection(s) between the control unit 110, which faces opposite the base portion 122, and the base portion 122 of the vibration module 104.

The rotatable mechanical indexing mechanism 128 may allow manual rotation of the vibration module 104 attached to the wearable band 102 to optimize alignment with the muscles or muscle groups of the body portion of the user, to direct and/or orient the vibrations in desired directions, and to ensure effective therapeutic stimulation to the targeted areas. The rotatable mechanical indexing mechanism 128 operates by allowing the vibration module or actuator to be rotated between multiple discrete angular positions about an axis, while providing a mechanical interaction that resists unintended rotation once a position is selected. During adjustment, rotation of the vibration module relative to the wearable band may align the vibration output along a desired direction or axis corresponding to a target body portion. The rotatable mechanical indexing mechanism 128 provides repeatable positional alignment, such that each selected rotational position corresponds to a known orientation of the vibration actuator(s). Once positioned, the indexing mechanism mechanically maintains the selected orientation during use, including while the user moves, thereby preventing drift or misalignment caused by body motion or vibration forces.

This indexed rotational positioning works in cooperation with the adjustable connection between the vibration module and the wearable band to enable directed and multi-axis vibration therapy, while avoiding the need for continuous manual repositioning or electronic orientation control.

In some embodiments, the wearable vibration therapy apparatus 100 may enable arrangement of one or more vibration actuators 112 so that the one or more vibration actuators 112 can be spaced around an area of the body portion of the user. For example, one or more vibration actuators 112 may be spaced around a knee joint or along a muscle group, using the rotatable mechanical indexing mechanism 128 to provide effective 360-degree or omnidirectional vibratory stimulation.

Referring now to FIG. 3, a vibration module 104 is shown detached from the control unit 110 of the wearable vibration therapy apparatus 100, in accordance with an embodiment of the present disclosure. The control unit 110 may be encased in a protective housing that encloses various electronic components of the apparatus 100. As shown in FIG. 1, the electronic components may include a processor 111 or a microcontroller and a memory 113. The memory 113 stores program instructions executable by the processor 111 and sensor data logs acquired during operation of the apparatus 100. The memory 113 may include both volatile and non-volatile memory components to support both temporary tasks and long-term data storage.

As shown in FIGS. 1 and 2, the control unit 110 may further include a motor driver circuit 115, which is electrically coupled to the one or more vibration actuators 112 located within the vibration module 104. The motor driver circuit 115 regulates the operation of the vibration actuators 112 based on control signals generated by the processor 111. Additionally, the control unit 110 includes one or more sensor interfaces for receiving real-time data from connected sensor modules, specifically the motion sensing module 106 and the tension sensing module 108. The sensor interfaces allow the processor to analyze motion and tension data received in real-time and adjust the operations of the apparatus 100 including the vibration parameters accordingly. Any of the sensor modules or other components of therapy apparatus 100 may include one or more sensors. Examples of such sensors include, but are not limited to: accelerometers, gyroscopes, pressure sensors, force sensors, temperature sensors, image sensors (e.g., Complementary Metal Oxide Semiconductor (CMOS) sensors, Charge-Coupled Device (CCD) sensors, etc.), light sensors, Photoplethysmography (PPG) sensors, impedance sensors, electrophysiological signal sensors, sensors gathering heart function data, sensors gathering sleep data, ultrasound or other visualization elements, flow sensors, motion sensors, Light Detection and Ranging (LiDAR) elements, sensors based on Doppler effect, sensors measuring physiological signals (like heart rate or galvanic skin response), sensors measuring relaxation parameters (e.g. heart rate variability HRV), etc. One or more sensors may be used to track movements of one or more elements of apparatus 100. This movement/positioning data may be used for determining orientation relative to anatomy, for documentation, for training, for guiding subsequent procedures, for alerting a user of correct or incorrect use, for alerting a user of risks, etc.

In some embodiments, the processor 111 within the control unit 110 may execute software instructions to carry out the following operations optionally including storing real-time sensor data received from the motion and/or tension sensing modules; retrieving historical usage data including preset personalized vibration therapy settings and/or vibration therapy parameters associated with a user; and/or executing a machine learning model to update the preset personalized vibration therapy settings of the user based on both current and historical data and/or dynamically control at least one vibration parameter, such as vibration intensity, vibration frequency, or vibration pattern, based on a combination of real-time data and updated personalized settings such as vibration parameters of the user. The vibration frequency of any of the embodiments disclosed herein may range from about 15 Hz to about 400 Hz. In some embodiments, vibration frequencies may fall within one or more subranges of this range, including but not limited to: about 15 Hz to about 25 Hz, about 20 Hz to about 40 Hz, about 30 Hz to about 60 Hz, about 40 Hz to about 80 Hz, about 60 Hz to about 100 Hz, about 80 Hz to about 120 Hz, about 100 Hz to about 150 Hz, about 120 Hz to about 180 Hz, about 150 Hz to about 220 Hz, about 180 Hz to about 260 Hz, about 200 Hz to about 300 Hz, about 250 Hz to about 350 Hz, and about 300 Hz to about 400 Hz.

In some embodiments, the vibration therapy apparatus generates vibrations having a frequency selected from overlapping subranges within about 15 Hz to about 400 Hz, including about 15 Hz to about 30 Hz, 20 Hz to about 50 Hz, 30 Hz to about 70 Hz, 50 Hz to about 90 Hz, 70 Hz to about 120 Hz, 90 Hz to about 150 Hz, 120 Hz to about 200 Hz, 150 Hz to about 250 Hz, 200 Hz to about 300 Hz, 250 Hz to about 350 Hz, and 300 Hz to about 400 Hz, with each subrange optionally selectable based on therapeutic application, target tissue, and user-specific parameters. In some embodiments, frequency ranges below about 15 Hz may be used. In some embodiments, frequency ranges above about 400 Hz may be used.

In some embodiments, the vibration therapy apparatus may be configured to operate within any individual frequency value, subrange, or combination of subranges within the range of about 15 Hz to about 400 Hz. In further embodiments, the vibration frequency may be dynamically adjusted in real-time within one or more of the foregoing subranges based on real-time sensor data and/or preset personalized vibration therapy settings associated with a user.

Some examples of therapy targets and their corresponding frequency ranges include, but are not limited to: surface tactile receptors (Merkel disks) targeted using about 5 Hz to about 15 Hz vibrations, mid-skin receptors (Meissner corpuscles) targeted using about 20 Hz to about 50 Hz vibrations, deep tissue Pacinian corpuscles targeted using about 60 Hz to about 400 Hz vibrations. One or more devices disclosed herein may have a frequency sweep or selection function to selects any of the frequency ranges disclosed herein. In some embodiments, about 250 Hz to about 500 Hz vibration is used for deeper penetration into soft tissue. In some embodiments, frequencies above about 1,000 Hz are used for targeting skin or other tissue surfaces.

In some examples, the casing or housing of the control unit 110 may include physical interface elements 124, such as a power switch, mode selection buttons, and indicator elements (e.g., LEDs), which may be used to display operational states of the apparatus 100 or provide status alerts. These components enhance usability by allowing the user to interact with the apparatus 100 directly using the physical user interface element(s) 124 in direct communication with the control unit 110 without the need for an external device. Any of the user interfaces described herein may be used to obtain feedback from the user such as pain relief felt, effectiveness of the vibration parameters, pain or discomfort felt, etc. Such feedback may be used as an input to modify one or more vibration parameters.

In some embodiments, the control unit 110 may include a communication module 114 (see, e.g., FIG. 1) for wireless connectivity, such as a Wi-Fi or Bluetooth Low Energy (BLE) radio. The wireless communication module 114 enables communication with external electronic devices, such as smartphones, smartwatches, tablets, remote computers, or other wearable devices. The wireless communication functionality allows the user to monitor the apparatus 100 remotely, view real-time sensor data, adjust vibration settings through a mobile application, receive firmware updates, and/or synchronize data related to the vibration therapy to an external server or cloud system for further analysis.

The control unit 110 may also generate one or more alerts or automatically adjust the tension of the wearable band 102 based on real-time feedback from the tension sensing module 108. This feature is particularly advantageous for ensuring optimal contact between the vibration module 104 and the body portion of the user during a vibration therapy session, thereby maintaining effective delivery of the therapeutic vibrations to the body portion.

In another embodiment, the control unit 110 may include a graphical user interface (GUI) or display 118, such as a touchscreen device, which is capable of presenting information including current vibration parameters, such as intensity, frequency, and pattern; battery status; and/or session progress. The user interface also allows the user to input adjustments to therapy parameters in real-time or update the preset personalized vibration therapy settings or other personalized settings such as the desired tension of the wearable band 102 for each body portion.

The control unit 110 may further distinguish vibrations generated by the vibration module 104 from the voluntary or involuntary body movements of the user, which further improves the accuracy and efficiency of the vibration therapy sessions. The control unit 110 differentiates the vibrations using frequency filtering algorithms or movement pattern recognition techniques applied to data received from the motion sensing module 106. This ensures that the control unit 110 does not misinterpret body movement as vibration feedback, which might otherwise lead to erroneous parameter adjustments. For example, the control unit 110 may distinguish vibrations generated by the vibration module 104 from voluntary or involuntary body movements by analyzing characteristics of sensor data received from the motion sensing module 106 over time. During operation, the motion sensing module 106 produces signals that include both vibration-induced components and movement-induced components. The control unit 110 applies frequency filtering algorithms to the received sensor data to isolate frequency bands corresponding to vibration frequencies generated by the vibration module 104, while attenuating lower-frequency or non-correlated signal components associated with body motion. In addition, or alternatively, the control unit 110 may apply movement pattern recognition techniques to identify signal patterns indicative of user movement, posture changes, or external motion, and differentiate such patterns from vibration signatures generated by the vibration module 104. Based on this differentiation, the control unit 110 selectively uses vibration-related sensor data for feedback control while disregarding or compensating for motion-related data, thereby preventing misinterpretation of body movement as vibration feedback and avoiding erroneous adjustment of vibration parameters.

Additionally, the control unit 110 may include a mode selection switch or virtual control that allows the user to manually cycle through or activate one of several preset vibrating modes. These modes may include therapy patterns optimized for relaxation, stimulation, muscle recovery, or warm-up routines, allowing users to personalize their therapy experience based on real-time needs or recommendations by an expert.

In yet another embodiment, the control unit 110 may be programmed to gradually reduce at least one vibration parameter, such as intensity or frequency, following the completion of a vibration therapy session. This gradual reduction of the vibration parameter allows the body part of the user to settle and adapt to the cessation of vibration, which can help minimize post therapy discomfort and contribute to a more comfortable recovery period.

In some embodiments, the motion sensing module 106 in communication with the control unit 110 is an IMU sensor. The motion sensing module 106 may include a 3-axis accelerometer and 3-axis gyroscope and, in some implementations, a magnetometer. The motion sensing module 106 continuously, or intermittently or on demand, measures an orientation of the apparatus 100 and quantitatively measures movements and/or vibrations experienced by the apparatus 100 during the movement of the user. The data thus collected using the motion sensing module 106 allows the control unit 110 to distinguish between the vibrations caused by the movement of the user and the vibrations produced by the vibration module 104.

For example, if the user starts walking while wearing the apparatus 100, the accelerometer may detect additional motion beyond the therapeutic vibrations, for example vibration in another frequency band. The control unit 110 can adjust a vibration parameter such as vibration intensity, vibration frequency, and/or vibration pattern to compensate for the change in vibrations due to the movement of the user. The motion sensing module 106 also continuously, or intermittently or on demand, and automatically verifies that the user receives vibrations with the intended or preset personalized vibration therapy settings. In one implementation, the data collected using the motion sensing module 106, especially the accelerometer may be used as feedback to measure the actual output vibration parameters such as vibration amplitude, frequency of vibrations, and/or vibration patterns. This ensures the consistency and accuracy of the therapeutic vibrations as input into the apparatus 100.

In some embodiments, the housing or casing at least partially enclosing the electronic components and the control unit 110 and the housing or casing of the vibration module 104 may be made from lightweight, impact-resistant material such as Acrylonitrile Butadiene Styrene (ABS) or polycarbonate plastic and, in some implementations, with a silicone or rubber outer layer for shock absorption and comfort. In some implementations, the electronic components of the control unit 110 and the vibration actuators 112 may be at least partially enclosed in protective housings that are either embedded in the wearable band 102 or attachable to, or incorporated into, the wearable band 102 as one or more modules.

In some embodiments, the housings may comprise or be made of materials that are hypoallergenic, breathable, and/or can withstand the vibrations generated and other external factors such as moisture. In designs described herein, i.e., by distributing the weight of the electronics and actuators within the wearable band 102, the apparatus 100 remains comfortable during use while maintaining good contact with the body portion of the user. Further, an inner surface of the wearable band 102 may be provided with a silicone grip pattern to prevent slipping and to evenly distribute pressure, while also being easy to clean.

The wearable vibration therapy apparatus 100 can be manufactured in different shapes and sizes appropriate for various body parts, all sharing the same or similar components and operating method.

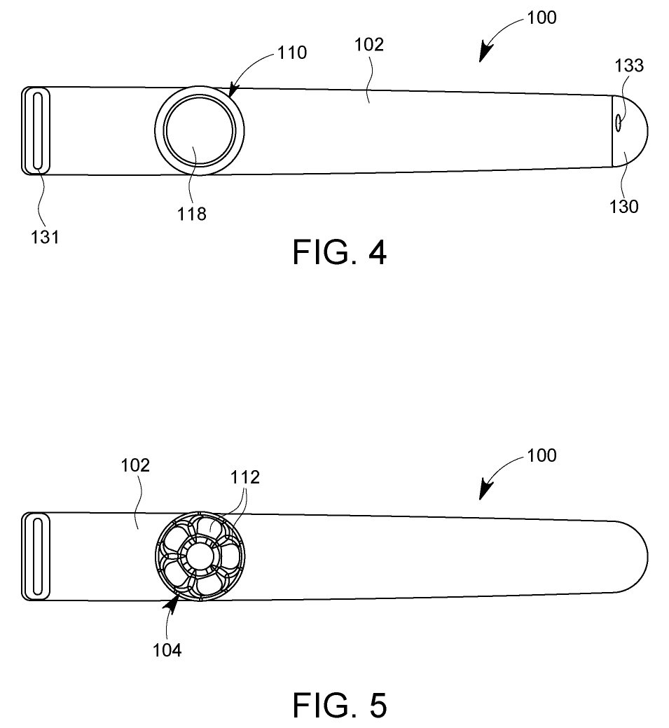

FIG. 4 illustrates a top view and FIG. 5 a bottom view of a wearable vibration therapy apparatus 100, according to an embodiment. In some embodiments, the wearable vibration therapy apparatus 100 with the wearable band 102 can be wrapped around a target body portion. Some examples of body portions that may be targeted using one or more embodiments include, but are not limited to: thigh, calf, arm, torso, neck, head, back (e.g. a lower back region, an upper back region, etc.), chest, an abdominal region, a pelvic region, and the like of the user. In some embodiments, apparatus 100 may be attached to one or more body portions with the help of an adjustable fastening means 130, 131 such as one or more hook-and-loop fasteners, with a wide range of adjustment allowable. The wearable band 102 may be made of flexible, durable materials such as neoprene or a nylon-elastic blend that conforms to and comfortably fits onto the body portion of the user. One or more of the components or elements described herein may be at least partially embedded in a portion of the wearable band 102.

In some embodiments, the tension sensing module 108 may be integrated into the wearable band 102. In some embodiments, the tension sensing module 108 may use a sensor 133, for example, a force sensor or load cell, at the anchor point of the strap (e.g. within a buckle or fastening clip, shown by fastening means 130, 131 in FIG. 4) that measures the force exerted by the tightened strap or the wearable band 102. In some embodiments, the wearable band 102 may use a stretch sensor for example, a strain gauge or piezoresistive elastic element woven into the band 102 that changes its electrical resistance based on how much the band 102 is stretched. In some embodiments, wearable band 102 or other components of apparatus 100 comprises one or more motorized tensioners. Such tensioners may be adjusted manually or through signals from control unit 110 or other components described herein. In some embodiments, such tensioners may be adjusted until a predetermined tension of wearable band 102 is reached. In some embodiments, adjustment of a tension of wearable band 102 or other components of apparatus 100 may be carried out to ensure consistent and/or maximum energy transfer of the vibrations from the source to the body portion. In some embodiments, such adjustments may be carried out to optimize the mechanical coupling of one or more components of apparatus 100 and a bodily region. A tension of wearable band 102 or other components of apparatus 100 may be adjusted to prevent wearable band 102 from being too loose (which prevents efficient transfer of vibrations to the body) or too tight (which may restrict blood flow). In some embodiments, wearable band 102 or other components of apparatus 100 includes one or more tension sensors (e.g., strain gauges, sensors based on monitoring current through motors, etc.) to measure a tension of wearable band 102 or other components of apparatus 100. Tightening or loosening of wearable band 102 or other components of apparatus 100 and/or adjustment of one or more vibration parameters may be performed using a closed-loop system with automatic adjustment of tension of wearable band 102 or other components of apparatus 100 and one or more vibration parameters. The target levels of a tension of wearable band 102 or other components of apparatus 100 may be determined using one or more stored settings.

In some embodiments, the tension sensing module 108 may provide a measurement correlating to how tight or snug the strap is, i.e., the wearable band 102 is on the body portion of the user. The control unit 110 may process the measurement data in real-time to gauge the tension of the wearable band 102 in real-time. This further enables the control unit 110 to detect if the wearable band 102 loosens during use or if the wearable band 102 was initially fastened too loosely or too tightly to the body portion. Further, the sensitivity of the tension sensing module 108 can be calibrated anytime by the user to achieve an optimal fit. The control unit 110 may record this value as part of the preset personalized vibration settings associated with the user. This allows the control unit 110 to dynamically adjust the vibration parameters based on the real-time movement and variations in tension of the wearable band 102 so that the user receives vibrations aligned with both the desired vibration parameters and the optimal fit of the wearable band 102, as determined by the preset personalized therapy settings.

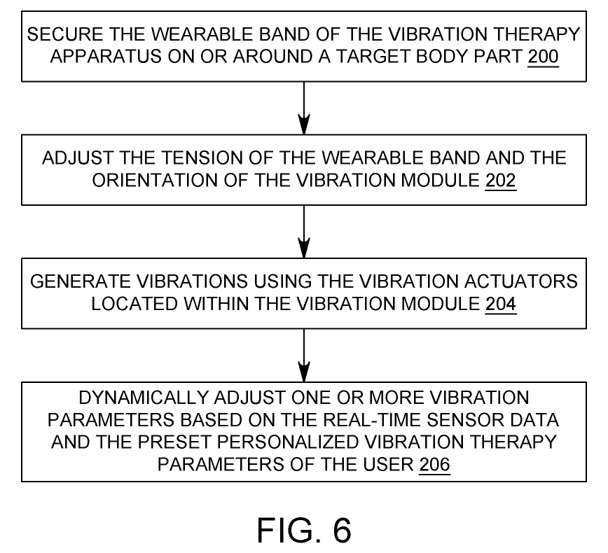

Referring now to FIG. 6, there is illustrated a flow diagram depicting a method for operating the wearable vibration therapy apparatus 100 to provide personalized vibration therapy to a user, in accordance with an aspect of the present disclosure. The method begins at block 200, where the wearable band 102 of the vibration therapy apparatus 100 is secured on or around a target body portion. The wearable band 102 may be designed to conform snugly to various body regions such as arms, legs, or joints. Proper placement of the wearable band 102 ensures that the therapeutic vibrations generated by the apparatus 100 are directed to the desired anatomical location.

At block 202, a tension of the wearable band 102 and/or the orientation of the vibration module 104 may be adjusted. Block 202 may be used to align the generated vibrations with the direction of stimulation desired for a therapy session. Proper tension of the wearable band 102 ensures consistent contact between the vibration module 104 and the body portion, while correct orientation of the vibration module 104 allows the vibrations to be targeted toward specific muscle groups or tissues for improved therapeutic outcomes. The adjustment may be performed manually, aided by visual feedback through the display 118 or preset alignment indicators, or automatically using tension sensing and actuator control features of the apparatus 100.

At block 204, the apparatus 100 generates vibrations using the vibration actuators 112 located within the vibration module 104. The vibrations may be dynamically controlled based on real-time sensor data received from the motion sensing module 106 and the tension sensing module 108, in combination with one or more preset personalized vibration therapy settings stored in the control unit 110. The apparatus 100 can integrate real-time feedback and/or stored preferences to ensure a highly personalized and adaptive therapeutic session that evolves in response to the movement of the user or a fit of the wearable band or strap 102.

In a further embodiment, the method may include continuously, or intermittently or on demand, detecting the movement and/or orientation of the wearable band 102 using the motion sensing module 106 and monitoring the tension of the band using the tension sensing module 108. This continuous monitoring process allows the control unit 110 to assess the real-time physical interaction between the apparatus 100 and the body portion, enabling real-time adjustments that maintain therapy effectiveness even during user movement. Although several embodiments described herein are based on adjusting the tension measured on wearable band 102 or other regions of apparatus 100, similar embodiments may be based on adjusting the force and/or pressure exerted by one or more device components (e.g., vibration module 104, wearable band 102, etc.) on one or more bodily regions.

The method may also include block 206 that recites dynamically adjusting one or more vibration parameters based on the real-time sensor data and/or the preset personalized vibration therapy settings associated with a user. Examples of vibration therapy settings or parameters include, but are not limited to: vibration intensity, frequency, location of vibration, direction of vibration, and vibration pattern. In any of the embodiments described herein, one or more vibration therapy settings or parameters may be varied by the user and/or the apparatus 100 through the course of a use session or therapy. In some embodiments, location of vibration and/or direction of vibration may be chosen by a selected one or more vibration modules 104 through which vibration therapy is delivered and/or selecting vibration therapy parameters of such vibration modules 104. The dynamic adjustments ensure that the therapy remains aligned with the therapeutic goals and user comfort, regardless of changes in posture, activity, or strap tension on the body portion of the user. Additionally, the method may include automatically logging vibration therapy session data, which includes sensor data, control signals, and outcome metrics. This logged data is used for improving future vibration therapy sessions.

In another embodiment, the method may include automatically alerting the user if deviations are detected from a preset tension range or if the real-time data deviates from the preset personalized vibration therapy settings. These alerts may be provided through visual indicators on the display 118 through the control unit 110, notifications on a paired computing device, or audible tones. Alerts enable the user to take corrective action, thereby ensuring the therapeutic session remains within optimal conditions.

In a further refinement, the method may include automatically adjusting the tension of the wearable band 102 and/or updating the preset personalized vibration therapy settings and/or vibration therapy parameters based on the real-time sensor data. These automated adjustments improve usability and safety, reducing the need for user intervention during therapy sessions and helping maintain optimal contact and vibration effectiveness.

In some embodiments, the method may incorporate the use of a machine learning model to analyze the logged vibration therapy session data. The system uses this analysis to update and refine the personalized vibration parameters over time. By learning from user-specific trends and historical outcomes, the apparatus 100 can provide more targeted, efficient, and effective vibration therapy in subsequent sessions.

The method may include a post-therapy routine where the control unit 110 gradually reduces the vibration intensity, frequency, or pattern after the completion of a session. This gradual reduction, or ramp-down, allows the body portion of the user to adjust smoothly from therapy to rest, enhancing comfort and supporting recovery. The controlled termination of the vibration ensures that muscle relaxation and blood circulation continue in a natural way, contributing to the overall therapeutic benefits of the apparatus 100.

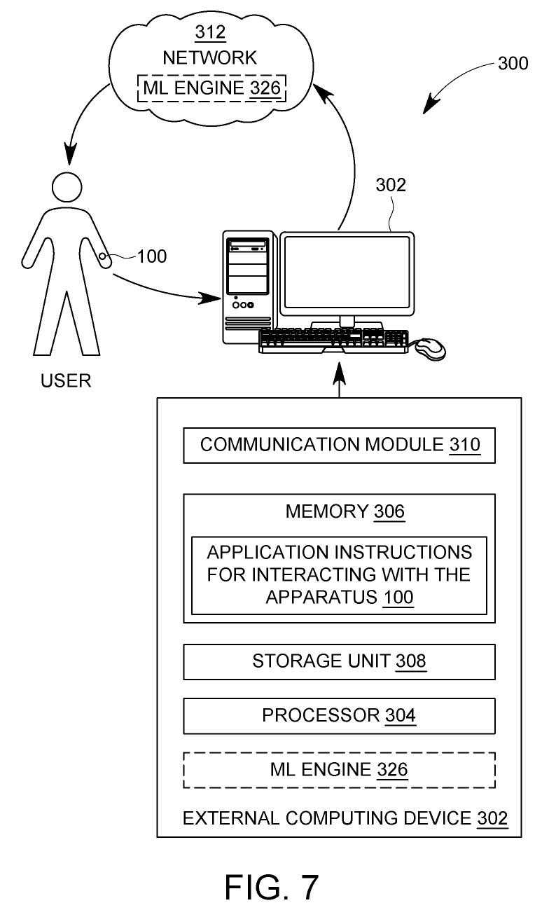

Referring now to FIG. 7, which shows a block diagram of a vibration therapy system 300 incorporating any of the embodiments of a wearable vibration therapy apparatus 100, in accordance with an aspect of the present disclosure. The system 300 is designed to enable enhanced control, monitoring, and/or optimization of vibration therapy by facilitating communication between the wearable vibration therapy apparatus 100 and one or more external computing devices. As shown in FIG. 7, the vibration therapy system 300 may include a vibration therapy apparatus 100, which includes a wearable band 102 that can be securely positioned on or around a body portion of a user and a vibration module 104 adjustably connected to the wearable band 102 for generating therapeutic vibrations. A motion sensing module 106 may be attachable to, or incorporated into, the wearable band 102 and may detect at least one of a movement or orientation of the wearable band 102. A tension sensing module 108 may be attachable to, or incorporated into, the wearable band 102 attached and may measure the tension of the wearable band 102. A control unit 110 may be attachable to, or incorporated into, the wearable band 102 and communicatively coupled to the vibration module 104, motion sensing module 106, and/or the tension sensing module 108. The control unit 110 may receive real-time sensor data and process these data together with preset personalized vibration therapy settings stored within the control unit 110 to generate control signals used to dynamically adjust at least one vibration parameter, such as vibration intensity, frequency, and/or pattern of vibrations applied to the body portion of the user.

The vibration therapy system 300 may further include an external computing device 302, which may communicate with the wearable vibration therapy apparatus 100 using a wired or wireless communication channel (e.g., Wi-Fi, Bluetooth low energy, etc.). The external computing device 302 may include a processor 304, a memory 306 that stores the instructions of a software application for interacting with the apparatus 100, a storage unit 308 for storing data received from the vibration therapy apparatus 100, and a communication module 310 to enable communication with the apparatus 100. Upon execution of the stored instructions, the processor 304 may perform several operations, such as display one or more vibration parameters e.g., vibration intensity, frequency, pattern, and/or real-time sensor data through a graphical user interface and process the real-time sensor data and/or previously stored sensor data or historical data associated with a user. The processor 304 may remotely adjust one or more vibration parameters and/or the preset personalized vibration therapy settings by transmitting updated control signals to the wearable apparatus 100. The processor 304 may further optimize the vibration parameters over time using an adaptive control model that analyzes a combination of real-time and historical sensor data along with the stored preset personalized vibration therapy settings of each of the users to adapt the therapy to an individual user.

In some embodiments, the external computing device 302 may continuously, or intermittently or on demand, monitor real-time sensor data during active therapy sessions and remotely control the vibration parameters, including vibration intensity, vibration frequency, and/or vibration patterns. This capability allows a healthcare provider, therapist, or even the user to dynamically modify the therapy settings (in some embodiments in real-time) to better suit the ongoing physical condition or comfort level of the user. Such real-time intervention supports immediate corrective action in case of suboptimal contact or sensor readings.

In another embodiment, the external computing device 302 may store data from one or more therapy sessions, including but not limited to, real-time sensor readings, control parameters, vibration parameters, user preferences, user feedback, session duration, and/or outcomes. These historical data may be used to track user progress over time. Based on long-term usage trends and previous therapy effectiveness, the system 300 can automatically adjust vibration parameters or update personalized settings for future sessions. The use of such adaptive algorithms significantly improves the continuity and precision of therapy while reducing the need for manual reconfiguration.

The integration of the wearable vibration therapy apparatus 100 with the external computing device 302 forms a comprehensive personalized vibration therapy system 300 that supports both autonomous operation and therapist-guided adjustments. The system 300 can leverage artificial intelligence and remote accessibility to deliver consistent, adaptive, and/or user-specific vibration therapy. The integration also enables secure data synchronization, remote diagnostics, and firmware updates, further expanding the effectiveness of therapeutic services using the wearable vibration therapy apparatus 100.

In some embodiments of the present disclosure, the wearable vibration therapy apparatus 100 employs an adaptive control method integrated into the control unit 110 to optimize vibration therapy in real-time. The adaptive control method is based on continuous feedback from the motion sensing module 106 or IMU sensor and the tension sensing module 108, allowing the apparatus 100 to dynamically adjust outputs of the vibration actuators 112 to match desired or preset therapeutic settings or outcomes or effectiveness. In some embodiments, control unit 110 executes these operations through embedded firmware instructions within the processor of the control unit 110, following structured, rule-based decision logic that may be fully embedded in hardware and software, thereby eliminating dependence on user intuition or manual intervention.

In one implementation, the adaptive control method operates within the system 300 that includes the external computing device 302 and the wearable vibration therapy apparatus 100. The adaptive control methodology may be implemented in a staged manner. In an initial calibration stage, the control unit 110 may read sensor data from the tension sensing module 108 to verify that the wearable band 102 is secured within a predefined tension range. The predefined range is defined by preset threshold values corresponding to a snug, but not overly tight, fit.

In some embodiments, the wearable band may operate within a tension range sufficient to maintain consistent mechanical coupling between the vibration module and a target body portion of a user while remaining comfortable during use. By way of non-limiting example, the wearable band may apply a tensile force within a range of about 0.5 newtons (N) to about 20 N, such as about 1 N to about 15 N, or about 2 N to about 10 N, depending on the body portion, band width, and material properties of the wearable band.

In some embodiments, the tension sensing module 108 monitors the applied tension and provides real-time tension data to the control unit 110, which determines whether the detected tension falls within a predefined range associated with preset personalized vibration therapy settings. If the detected tension falls below the predefined range associated with effective vibration transmission or exceeds the predefined range associated with user comfort, the control unit 110 may generate an alert and/or adjust one or more vibration parameters to maintain effective and/or comfortable therapy delivery.

In some embodiments, visual indicators such as LEDs or wireless feedback using a paired computer application can prompt the user to adjust the wearable band 102 accordingly. Once the appropriate fit is detected, the system 300 may store the baseline tension value in the control unit 110 or in the system memory. Substantially simultaneously, or within a predefined time window, the motion sensing module 106 may capture baseline orientation and/or acceleration data to account for gravitational effects and/or static offsets. This reference calibration ensures that subsequent movement and vibration detection are interpreted relative to a consistent starting condition.

During a vibration therapy session execution stage, the system 300 and the wearable vibration therapy apparatus 100 may transition into an active therapy mode. The control unit 110 may initiate activation of the vibration module 104 using either a user-selected program e.g., "muscle warm up", "pain relief", or a default setting. Each program may define a distinct combination of vibration intensity, frequency, and/or pattern. If the user has not selected a specific mode, the system 300 or the wearable vibration therapy apparatus 100 may select a default moderate, balanced setting and gradually increase or decrease the vibration parameters based on a response of the user. The control unit 110 may drive the one or more vibration actuators 112 accordingly, referencing the initial calibration data and any preset personalized vibration settings stored in the memory, to achieve the intended therapy effectiveness or result.

The motion sensing module 106 may monitors accelerometer signals to measure the actual vibration output and identify movement of the body portion of the user. If the vibration actuators 112 are vibrating at, for instance, 30 Hz, the system 300 or the apparatus 100 may expect to detect this frequency in the collected real-time sensor data as feedback. Any deviation, which may be caused by motion, poor contact, or misalignment, can be identified and filtered using a frequency recognition or cross-referenced using gyroscope data. This ensures that unintended movements (e.g., walking, posture changes, etc.) are not misinterpreted as vibration inconsistencies. The control unit 110 differentiates between these signals in real-time and responds accordingly.

Based on this real-time feedback, the control unit 110 may perform dynamic adjustments to one or more vibration parameters. For example, if reduced amplitude is detected, potentially due to the wearable band 102 loosening or shifting from the target muscle group, the apparatus 100 may increase actuator power or lower the frequency to improve penetration. Similarly, if erratic movement is detected that suggests bouncing or poor contact, the apparatus 100 may decrease intensity or pause vibration operation while alerting the user. If the user is actively exercising while wearing the apparatus 100, the adaptive control method may switch to a lower-intensity mode to avoid interfering with the activity while still providing some therapeutic vibration.

The tension sensing module 108 of the apparatus 100 may monitor the tension of the wearable band 102, and the respective feedback loop may run continuously throughout each vibration therapy session (or intermittently in some embodiments). The tension sensing module 108 may track a tightness of the wearable band 102 and flag deviations from a baseline or predefined range. If a drop in tension is detected, which may be due to sweat, slippage, or muscle contraction, the apparatus 100 may generate an alert or, in certain embodiments, engage an active tensioning mechanism. Examples of such mechanisms include micro-motor winches, shape-memory alloy actuators, or pneumatic systems. If the strap or the wearable band 102 is overtightened, the apparatus 100 can recommend user adjustments or activate loosening mechanisms to maintain safe pressure levels. To prevent overcorrection, the apparatus 100 may implement hysteresis or time delays to avoid over-correcting for minor fluctuations.

Upon completion of a vibration session, the control unit 110 may perform a gradual ramp-down of the vibration intensity to facilitate muscle relaxation and minimize abrupt stimulus cessation. A post-session validation step including final tension and orientation checks may be performed by the control unit 110 and/or the system 300. The session data including vibration patterns used, dynamic adjustments made, sensor logs, and/or strap fit consistency, may be optionally stored locally in the memory associated with the apparatus 100 or the system 300 or transmitted wirelessly to a paired external computing device 302. This session data supports long-term therapy tracking and analysis, as well as refining the operation of the apparatus 100 by providing iterative updates to the personalized vibration parameters of each user using an appropriate adaptive control, personalized method for effective future vibration therapy sessions.

In some embodiments, the system employs an adaptive control method implemented in firmware, software, or a combination thereof, which operates based on physical measurements obtained from one or more sensor modules. During operation, the control unit 110 may monitor one or more measured vibration characteristics and compare the measured values to corresponding expected, target, or reference values associated with the current operating state of the vibration module. When a deviation between a measured vibration characteristic and an expected value satisfies one or more predefined conditions, such as exceeding a magnitude threshold and/or persisting for a predefined duration, the control unit 110 executes a corresponding control action. The control action may include adjusting one or more operating parameters of the vibration module, such as drive voltage, drive current, duty cycle, frequency, and/or waveform characteristics. In this manner, the adaptive control method enables closed-loop regulation of vibration output based on sensor feedback, while accommodating changes in coupling conditions, body movement, or system dynamics. By way of a non-limiting example, a predefined rule may state: "If measured vibration amplitude is 30% below expected value for more than 3 seconds, increase motor voltage by 10%." Such control logic ensures robust, real-time closed-loop therapy, minimizing user burden while maximizing adaptability and precision. The use of adaptive control methods in the control unit 110, or on an external computing device 302, enhances the adaptive capability of the system 300 or apparatus 100 by learning from session histories and optimizing future vibration settings.

The comprehensive adaptive control method allows the wearable vibration therapy apparatus 100 to respond effectively to real-world usage scenarios, providing motion-tolerant, tension-aware, and/or artificial intelligence based personalized therapy modes. The intelligent feedback loop ensures consistency in therapeutic delivery and supports a variety of use cases ranging from rehabilitation to athletic recovery and chronic pain management. The feedback loop works in real-time. The data from each session also helps improve future sessions, which makes the vibration therapy using the present apparatus 100 and system 300 smarter, more comfortable, and more effective over time.

Use of machine learning models to refine vibration therapy:

In some embodiments, as shown in FIGS. 1 and 7, the rule‑based adaptive control of the operations of the wearable vibration therapy apparatus 100 includes one or more machine‑learning (ML) models that refine the vibration therapy to each user over many sessions. The control unit 110 records real‑time sensor data and historical usage data, then runs an ML engine 326 operating one or more ML model to identify patterns in the way each user interacts with the apparatus 100. The ML engine 326 may operate on a network 312 (FIG. 7), locally on the apparatus 100 (FIG. 1), and/or on a communicatively coupled external computing device 302 (FIG. 7). In one example, the ML engine 326 may track how often a user raises vibration intensity for post‑workout recovery versus how often the same user selects a gentle setting for relaxation. From these patterns, the ML engine 326 can predict and preload a starting intensity, frequency, and/or pattern that better matches the habits of an associated user, rather than reverting to a generic default. In some embodiments, the vibration therapy session length may also be adjusted, which enables the apparatus 100 to stop or extend or modify a vibration therapy session in line with user behavior unless the automated therapy duration adjustment is overridden through the apparatus or an external electronic device 302 in communication with the apparatus 100.

Further, the ML engine 326 can also relate physiological response to specific vibration parameters. Data from the motion sensing module 106 and, in some embodiments, other health related data including heart‑rate or other health parameters of the user obtained using appropriate sensors, may provide indirect feedback on muscle relaxation or joint mobility. By correlating these signals with delivered frequency and amplitude, the ML engine 326 can determine, for example, that a 25 Hz vibration for 15 minutes yields the best pain‑relief outcome for a particular user, while higher frequencies add little benefit. Future sessions are then auto‑tuned to emphasize the most effective settings without further user input.

In an embodiment, a trained version of the ML engine 326 may employ reinforcement learning or small neural networks to fine‑tune the vibration pattern in real-time. During a session, the ML engine 326 may test slight changes in vibration parameters or settings of the apparatus 100 such as the tension of the wearable band 102 within safe limits and analyze the resulting sensor data or brief user feedback gathered through a companion application. Over successive sessions the ML engine 326 may converge on a control policy that maximizes comfort and therapeutic effect. For instance, the ML engine 326 may learn to trim vibration intensity after ten minutes, because tension data show that the band 102 tends to loosen at higher levels, or it may insert a short high‑frequency burst near the end of a program when past logs indicate improved pain scores.

The apparatus 100 can store multiple user profiles or usage modes. The ML engine 326 may distinguish among profiles, such as an athlete using a recovery mode and an office worker using a low‑back tension mode and applies the correct personalized parameters for each user. A therapist or the user can also declare a target outcome, like “reduce stiffness” or “improve circulation,” and the model may adjust its control strategy to favor vibration settings historically linked to that target outcome. To support these capabilities, the firmware may include compact ML libraries for data logging and on‑device inference. Heavier training tasks can be off‑loaded from the apparatus 100 to a paired external device 302 such as a smartphone or a cloud service (e.g., network 312). In such scenarios, the session data is sent to the external device 302, where a cloud‑based model processes the information and returns updated parameters to the control unit 110. The apparatus 100 may improve learning and applications with continued use, offering smoother, more effective sessions with minimal extra steps.

In some embodiments, ML based functions operate in addition to the deterministic safety controls already embedded in the firmware of the apparatus 100. Fail‑safes limit maximum intensity, frequency, and/or strap tension. If the ML model recommendation exceeds a safe threshold, the control unit 110 enforces the lower, predefined limit. In this way, the system 300 and the apparatus 100 deliver individualized effective vibration therapy while maintaining a transparent, rule‑based framework that preserves user safety and comforts captured from various viewpoints.

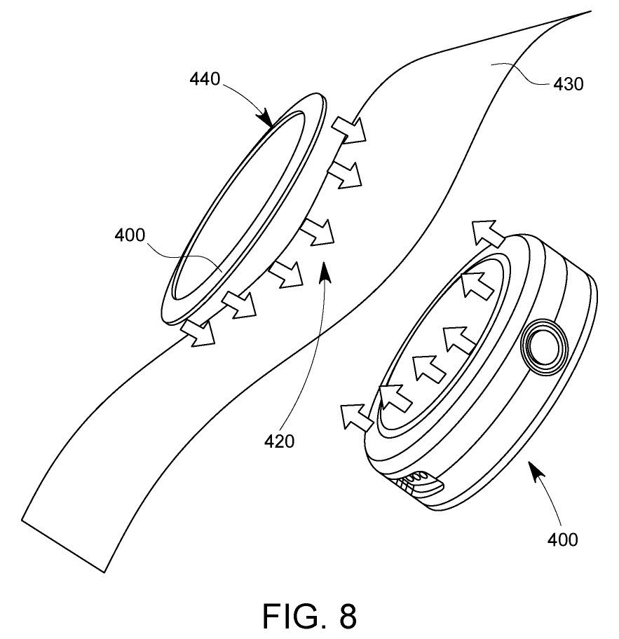

Referring now to FIG. 8, there is illustrated an embodiment of a therapy module being attached to a ring support base plate 410. In the embodiment shown, a PBM module 400 is shown being attached to an attachment site 420 using a magnetic attachment mechanism 440. Attachment site 420 includes any standard clothing material 430 that may support the attachment mechanism effectively for treatment. Although a ring-shaped base plate 410 is shown, one of skill in the art will appreciate that many shapes for the attachment base plate 410 is contemplated herein. For example, the base plate 410 could be in the shape of a disk, oval, square, rectangle, triangle, polygon, or other geometries that match the module profile. In addition, some base plate 410 designs could allow for more than one module attachment at a site such as a honey-comb ring base plate 410 design.

One advantage of a magnetic attachment mechanism 440 is that the user can easily attach or detach modules 400 in locations as desired. Designs of modules 400 are small and easily applied, thus they exert less pressure or force on the skin or bone. This makes them more comfortable and discreet during use. Any of the skin-contacting regions of one or more modules 400 disclosed herein may include concave surface(s) on a skin or body facing surface of the module 400 to better conform to the skin surface. The concave surface(s) may be tailored to fit the anatomical region where one or more modules 400 may be positioned.

Multiple attachment sites may be utilized simultaneously to cover the area of interest for treatment. The attachment mechanism and hold strength may be chosen so as to allow for a range of clothing materials and thicknesses to be worn without dislodging the module(s) during treatment.

An additional embodiment of the present disclosure may include an automatic turn-on/turn-off function which is activated upon attachment and detachment. Although it is envisioned that the user can have software control of the device functionality, a simpler methodology would allow for the aforementioned auto-feature for turn-on and turn-off if desired.

In addition to, or alternatively to, the embodiment of FIG. 8, any of the devices, systems, or features described in U.S. Patent Application Ser. No. 19/298,879, filed Aug. 13, 2025, which is herein incorporated by reference in its entirety, may also be included herein and/or combined with any of the vibration inducing systems and/or devices described herein.

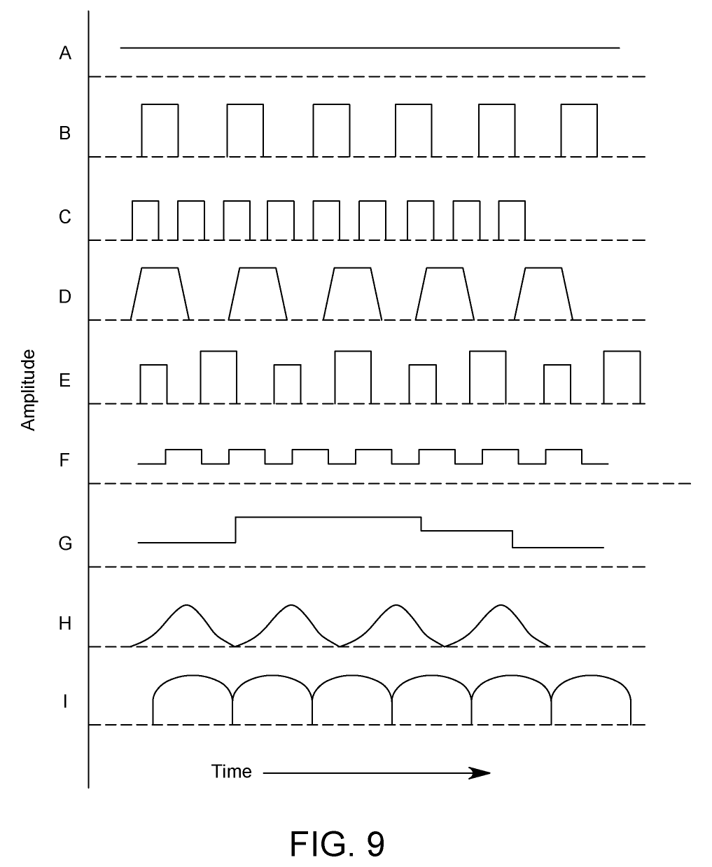

FIG. 9 shows some embodiments of vibration patterns that may be delivered through one or more embodiments of the present disclosure. The vertical axis shows the amplitude of the vibration, and the horizontal axis is time. Pattern A shows vibration being delivered continuously throughout the treatment. In this embodiment, the amplitude of the vibration is constant throughout. Pattern B shows an embodiment wherein vibration is delivered intermittently. Pattern B shows vibrations being turned on and off during the course of the therapy by vibration module 104. Pattern C also shows intermittent delivery of vibration. Pattern D shows intermittent delivery of vibration where the amplitude is gradually increased, held steady, and then gradually reduced. Pattern E shows vibration being delivered intermittently, where the amplitude of the vibration is varying during the therapy. Pattern F shows a continuous delivery of vibration, where the amplitude of the vibration is varying during the therapy. Pattern G shows a continuous delivery of vibration where the amplitude of the vibration is reduced towards the end of the therapy. Pattern H shows an embodiment where the amplitude of the vibration increases and reduces throughout the course of the therapy. Pattern I shows another embodiment where the amplitude of the vibration increases and reduces during the therapy. In any of the method embodiments, one or more parameters of the vibration such as the amplitude, frequency, vibration pattern, location of vibration, direction of the vibration, etc. may be varied during a therapy session. Various vibration patterns (some examples of which are shown in FIG. 9), may be combined during a therapy session. In some method embodiments, one or more vibration delivery parameters may be chosen by apparatus 100. In some embodiments, one or more vibration delivery parameters may be selected by the user through a user interface.

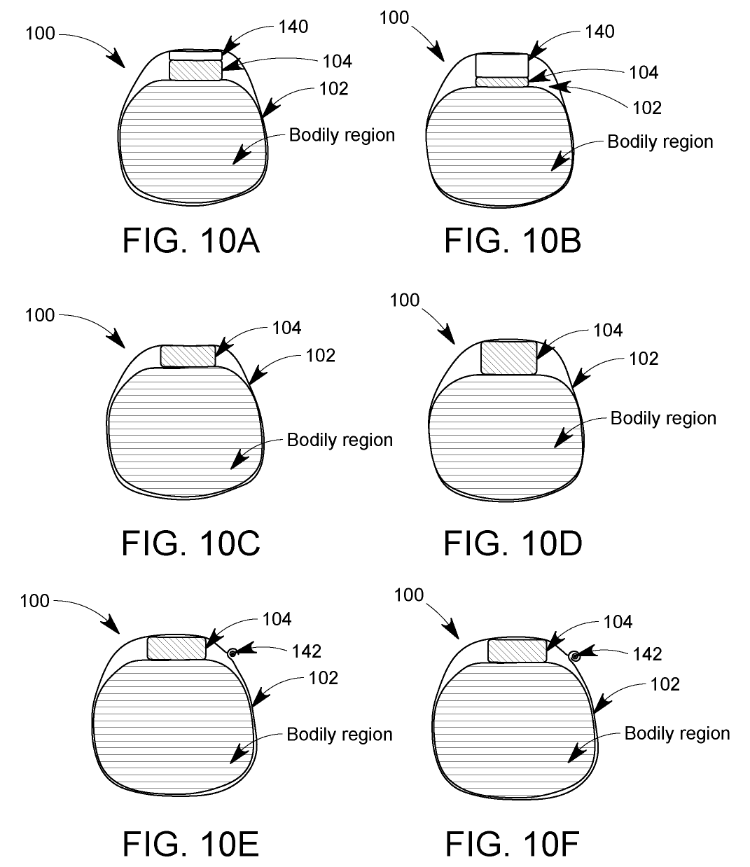

FIGS. 10A - 10F show various embodiments of devices and methods to adjust the contact force and/or pressure of one or more elements of therapy apparatus 100 against a bodily region. In some embodiments, the contact force and/or pressure may be adjusted by adjusting the tension on wearable band 102. In some embodiments, the contact force and/or pressure may be adjusted to deliver optimal therapy to the user. In some embodiments, the contact force and/or pressure may be adjusted to optimize user comfort as well as the efficacy of one or more therapies. In some embodiments, the contact force and/or pressure may be adjusted to ensure user safety.

FIG. 10A shows an embodiment of a vibration therapy apparatus comprising an inflatable element 140. Inflatable element 140 may be inflated using one or more fluids (e.g., gas, saline, gel, etc.). In some embodiments, inflatable element 140 may include one or more pneumatic actuators. An air pump or other fluid pump may be used to inflate inflatable element 140, as shown in FIG. 10B, to increase the force and/or pressure of vibration module 104 against a bodily region. An air pump or other fluid pump may be used to deflate inflatable element 140 to reduce the force and/or pressure of vibration module 104 against a bodily region. After adjusting the force and/or pressure, one or more vibration parameters may be determined and/or adjusted by therapy apparatus 100.

FIG. 10C shows an embodiment of a vibration therapy apparatus including an expandable vibration module 104. Vibration module 104 may include one or more motors, bendable elements, foldable elements, actuators, inflatable elements, etc. that allow vibration module 104 to be expanded or contracted. In some embodiments, other elements of therapy apparatus 100 may be expandable and contractable. In some embodiments, inflatable element 140 may include one or more pneumatic actuators. Vibration module 104 may be expanded, as shown in FIG. 10D relative to FIG. 10C, to increase the force and/or pressure of vibration module 104 against a bodily region. Vibration module 104 may be contracted to reduce the force and/or pressure of vibration module 104 against a bodily region. After adjusting the force and/or pressure, one or more vibration parameters may be determined and/or adjusted by therapy apparatus 100.

FIG. 10E shows an embodiment of a vibration therapy apparatus including a spooling mechanism. Spooling mechanism 142 may include one or more rotating rods around which one or more regions of wearable band 102 are wrapped. Rotating the rod(s) wraps or unwraps regions of wearable band 102 around the rod(s). In this way, the tension on one or more regions of wearable band 102 and hence the force and/or pressure of vibration module 104 against a bodily region may be adjusted. After adjusting the force and/or pressure, one or more vibration parameters may be determined and/or adjusted by therapy apparatus 100.

The systems and methods described herein, and variations thereof, can be embodied and/or implemented at least in part as a machine configured to receive a computer-readable medium storing computer-readable instructions. The instructions are executable by computer-executable components integrated with the system and one or more portions of the processor on the PBM device, vibration therapy module, and/or a computing device communicatively coupled to the PBM device, vibration therapy module, or a system thereof. The computer-readable medium can be stored on any suitable computer-readable media such as RAMs, ROMs, flash memory, EEPROMs, optical devices (e.g., CD or DVD), hard drives, floppy drives, or any suitable device. The computer-executable component may be a general or application-specific processor, but any suitable dedicated hardware or hardware/firmware combination can alternatively, or additionally, execute the instructions.

### Examples

Example 1. A vibration therapy apparatus comprising: a wearable band configured for placement on or around a body portion of a user; a vibration module adjustably connected to the wearable band and configured to generate vibrations; a motion sensing module attachable to the wearable band and configured to detect at least one of a movement or orientation of the wearable band; and a control unit attachable to the wearable band, the control unit being communicatively coupled to the vibration module and the motion sensing module, wherein the control unit is configured to: receive real-time sensor data from the motion sensing module, generate one or more signals based on the real-time sensor data and one or more preset personalized vibration therapy settings stored in the control unit, and based on the one or more signals, cause the vibration module to dynamically adjust at least one vibration parameter associated with the vibrations.

Example 2. The vibration therapy apparatus of any one of the preceding examples, but particularly Example 1, wherein the vibration module is configured to receive the one or more signals from the control unit to dynamically adjust the at least one vibration parameter comprising one or more of: a vibration intensity, a vibration frequency, or a vibration pattern.

Example 3. The vibration therapy apparatus of any one of the preceding examples, but particularly Example 1, wherein the vibration module comprises one or more modular vibration actuators, each configured to generate the vibrations in at least one direction, wherein a position of each of the one or more modular vibration actuators is adjustable.

Example 4. The vibration therapy apparatus of any one of the preceding examples, but particularly Example 3, wherein the one or more modular vibration actuators comprise two pairs of modular vibration actuators, wherein a first pair is positionable orthogonally relative to a second pair to generate the vibrations along orthogonal planes.

Example 5. The vibration therapy apparatus of any one of the preceding examples, but particularly Example 3, wherein at least one modular vibration actuators is a linear resonant actuator (LRA) or a pair of eccentric rotating mass (ERM) motors.

Example 6. The vibration therapy apparatus of any one of the preceding examples, but particularly Example 1, wherein the vibration module comprises one or more modular vibration actuators, wherein a position of a modular vibration actuator of the one or more modular vibration actuators is secured to the wearable band using a rotatable mechanical indexing mechanism.

Example 7. The vibration therapy apparatus of any one of the preceding examples, but particularly Example 1, wherein the vibration module is magnetically attached to the control unit.

Example 8. The vibration therapy apparatus of any one of the preceding examples, but particularly Example 1, wherein the motion sensing module comprises one or more of: an accelerometer or a gyroscope.

Example 9. The vibration therapy apparatus of any one of the preceding examples, but particularly Example 1, further comprising a tension sensing module attachable to the wearable band and communicatively coupled to the control unit, wherein the tension sensing module is configured to measure a tension of the wearable band.

Example 10. The vibration therapy apparatus of any one of the preceding examples, but particularly Example 9, wherein the control unit is further configured to: receive second real-time sensor data from the tension sensing module, and cause the tension sensing module to adjust the tension of the wearable band.

Example 11. The vibration therapy apparatus of any one of the preceding examples, but particularly Example 9, wherein the tension sensing module comprises a sensor selected from a list consisting of: strain gauges, stretch sensors, pressure sensors, force sensors, and load cells.

Example 12. The vibration therapy apparatus of any one of the preceding examples, but particularly Example 9, wherein the control unit is configured to generate at least one of: an alert or automatic adjustment of the tension of the wearable band

Example 13. The vibration therapy apparatus of any one of the preceding examples, but particularly Example 1, wherein the wearable band comprises a therapeutic pad, sleeve, or wrap configured to attach to the body portion of the user using an adjustable fastening means.

Example 14. The vibration therapy apparatus of any one of the preceding examples, but particularly Example 1, wherein the control unit further comprises: a processor, and a memory storing instructions executable by the processor, wherein execution of the instructions by the processor cause application of a machine learning model to dynamically control the at least one vibration parameter in real-time based on at least one of: the real-time sensor data and the one or more preset personalized vibration therapy settings.

Example 15. The vibration therapy apparatus of any one of the preceding examples, but particularly Example 1, further comprising a user interface configured to: display information including the at least one vibration parameter, and receive user inputs to adjust the at least one vibration parameter or the one or more preset personalized vibration therapy settings.

Example 16. The vibration therapy apparatus of any one of the preceding examples, but particularly Example 15, wherein the user interface comprises a touch screen device.

Example 17. The vibration therapy apparatus of any one of the preceding examples, but particularly Example 1, wherein the control unit is configured to distinguish the vibrations caused by the vibration module from a movement of the body portion of the user by applying frequency filtering or movement pattern recognition.

Example 18. The vibration therapy apparatus of any one of the preceding examples, but particularly Example 1, wherein the control unit comprises a mode selection switch to enable manual switching between a plurality of preset vibration modes.

Example 19. The vibration therapy apparatus of any one of the preceding examples, but particularly Example 1, wherein the control unit is configured to gradually reduce the at least one vibration parameter after a therapy session.

Example 20. The vibration therapy apparatus of any one of the preceding examples, but particularly Example 1, further comprising one or more photobiomodulation modules.

Example 21. A vibration therapy system comprising: a vibration therapy apparatus comprising: a wearable band configured for placement on or around a body portion of a user; a vibration module adjustably connected to the wearable band and configured to generate vibrations; a motion sensing module attachable to the wearable band and configured to detect at least one of a movement or orientation of the wearable band; a control unit attachable to the wearable band, the control unit being communicatively coupled to the vibration module and the motion sensing module, wherein the control unit is configured to: receive real-time sensor data from the motion sensing module, generate one or more signals based on the real-time sensor data and one or more preset personalized vibration therapy settings stored in the control unit, and based on the one or more signals, cause the vibration module to dynamically adjust at least one vibration parameter associated with the vibrations; and an external computing device configured to communicate with the vibration therapy apparatus, the external computing device comprising a processor, a memory storing instructions of an application, and a storage unit configured to store data received from the vibration therapy apparatus, wherein, the processor is configured to execute the instructions to cause the external computing device to: display the one or more of: the at least one vibration parameter, the real-time sensor data, or the vibration therapy apparatus data using a user interface, and remotely cause the control unit to execute one or more of: adjust one or more vibration parameters in real-time, adjust the one or more preset personalized vibration therapy settings, or optimize the one or more vibration parameters based on one or both of: the vibration therapy apparatus data or the real-time sensor data by applying a machine learning method.

Example 22. The vibration therapy system of any one of the preceding examples, but particularly Example 21, further comprising a tension sensing module attachable to the wearable band and communicatively coupled to the control unit, wherein the tension sensing module is configured to measure a tension of the wearable band.

Example 23. The vibration therapy system of any one of the preceding examples, but particularly Example 21, wherein the external computing device is configured to monitor the real-time sensor data and remotely control the one or more vibration parameters including a vibration intensity, a vibration frequency, or a vibration pattern during one or more therapy sessions.

Example 24. The vibration therapy system of any one of the preceding examples, but particularly Example 21, wherein the external computing device is configured to store data associated with one or more therapy sessions to track user progress and adjust the one or more vibration parameters or the one or more preset personalized vibration therapy parameters for future therapy sessions based on historical patterns.

Example 25. A device for delivering a photobiomodulation therapy, comprising: a base support; an attachment means for securing to a user; one or more module attachment sites for coupling to the base support; and one or more photobiomodulation modules adapted to reversibly attach to the one or more module attachment sites using the base support and through clothing material or garments.

Example 26. The device of any one of the preceding examples, but particularly Example 25, wherein the attachment means comprises one of: a magnet, a ferrous metal material, or a mechanical mating profile.

Example 27. The device of any one of the preceding examples, but particularly Example 25, wherein the one or more photobiomodulation modules comprise an automatic turn-on/turn-off function upon attaching to the base support.

Example 28. A method of providing vibration therapy, the method configured to be performed by a control unit and comprising: receiving real-time sensor data from a motion sensing module and a tension sensing module; generating one or more signals based on the received real-time sensor data and one or more preset personalized vibration therapy settings stored in the control unit; based on the one or more signals, causing: a vibration module to dynamically adjust at least one vibration parameter associated with vibrations generated by the vibration module, the tension sensing module to adjust a tension of a wearable band associated with the motion sensing module, the tension sensing module, and the vibration module, an orientation of the vibration module to align the vibrations along a desired direction to achieve the one or more preset personalized vibration therapy settings, and the vibration module to generate the vibrations consistent with the at least one vibration parameter and one or more present personalized vibration therapy settings.

Example 29. The method of any one of the preceding examples, but particularly Example 28, further comprising continuously detecting a movement and an orientation of the wearable band using the motion sensing module, and monitoring the tension of the wearable band using the tension sensing module to generate the real-time sensor data.

Example 30. The method of any one of the preceding examples, but particularly Example 28, further comprising dynamically adjusting the one or more vibration parameters based on the real-time sensor data and the one or more preset personalized vibration therapy settings and automatically logging each vibration therapy session data.

Example 31. The method of any one of the preceding examples, but particularly Example 28, further comprising automatically outputting an alert when deviations from a predefined tension range or the one or more preset personalized vibration therapy settings are detected.

Example 32. The method of any one of the preceding examples, but particularly Example 28, further comprising employing a machine learning method to analyze vibration therapy session data and update the one or more preset personalized vibration therapy parameters based on one or both of: the real-time sensor data or logged vibration therapy session data.

References in the specification to “one embodiment,” “an embodiment,” “an illustrative embodiment,” “some embodiments,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described.

As used in the description and claims, the singular form “a”, “an” and “the” include both singular and plural references unless the context clearly dictates otherwise. For example, the term “sensor” or “module” or “session” may include, and is contemplated to include, a plurality of sensors, a plurality of modules, or a plurality of sessions. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by ( + ) or ( - ) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A vibration therapy apparatus comprising:
a wearable band configured for placement on or around a body portion of a user;
a vibration module adjustably connected to the wearable band and configured to generate vibrations;
a motion sensing module attachable to the wearable band and configured to detect at least one of a movement or orientation of the wearable band; and
a control unit attachable to the wearable band, the control unit being communicatively coupled to the vibration module and the motion sensing module, wherein the control unit is configured to:
receive real-time sensor data from the motion sensing module,
generate one or more signals based on the real-time sensor data and one or more preset personalized vibration therapy settings stored in the control unit, and
based on the one or more signals, cause the vibration module to dynamically adjust at least one vibration parameter associated with the vibrations.

2. The vibration therapy apparatus of claim 1, wherein the vibration module is configured to receive the one or more signals from the control unit to dynamically adjust the at least one vibration parameter comprising one or more of: a vibration intensity, a vibration frequency, or a vibration pattern.

3. The vibration therapy apparatus of claim 1, wherein the vibration module comprises one or more modular vibration actuators, each configured to generate the vibrations in at least one direction, wherein a position of each of the one or more modular vibration actuators is adjustable.

4. The vibration therapy apparatus of claim 3, wherein the one or more modular vibration actuators comprise two pairs of modular vibration actuators, wherein a first pair is positionable orthogonally relative to a second pair to generate the vibrations along orthogonal planes.

5. The vibration therapy apparatus of claim 3, wherein at least one modular vibration actuators is a linear resonant actuator (LRA) or a pair of eccentric rotating mass (ERM) motors.

6. The vibration therapy apparatus of claim 1, wherein the vibration module comprises one or more modular vibration actuators, wherein a position of a modular vibration actuator of the one or more modular vibration actuators is secured to the wearable band using a rotatable mechanical indexing mechanism.

7. The vibration therapy apparatus of claim 1, wherein the vibration module is magnetically attached to the control unit.

8. The vibration therapy apparatus of claim 1, wherein the motion sensing module comprises one or more of: an accelerometer or a gyroscope.

9. The vibration therapy apparatus of claim 1, further comprising a tension sensing module attachable to the wearable band and communicatively coupled to the control unit, wherein the tension sensing module is configured to measure a tension of the wearable band.

10. The vibration therapy apparatus of claim 9, wherein the control unit is further configured to:
receive second real-time sensor data from the tension sensing module, and
cause the tension sensing module to adjust the tension of the wearable band.

11. The vibration therapy apparatus of claim 9, wherein the tension sensing module comprises a sensor selected from a list consisting of: strain gauges, stretch sensors, pressure sensors, force sensors, and load cells.

12. The vibration therapy apparatus of claim 9, wherein the control unit is configured to generate at least one of: an alert or automatic adjustment of the tension of the wearable band.

13. The vibration therapy apparatus of claim 1, wherein the wearable band comprises a therapeutic pad, sleeve, or wrap configured to attach to the body portion of the user using an adjustable fastening means.

14. The vibration therapy apparatus of claim 1, wherein the control unit further comprises: a processor, and a memory storing instructions executable by the processor, wherein execution of the instructions by the processor cause application of a machine learning model to dynamically control the at least one vibration parameter in real-time based on at least one of: the real-time sensor data and the one or more preset personalized vibration therapy settings.

15. The vibration therapy apparatus of claim 1, further comprising a user interface configured to:
display information including the at least one vibration parameter, and
receive user inputs to adjust the at least one vibration parameter or the one or more preset personalized vibration therapy settings.

16. The vibration therapy apparatus of claim 15, wherein the user interface comprises a touch screen device.

17. The vibration therapy apparatus of claim 1, wherein the control unit is configured to distinguish the vibrations caused by the vibration module from a movement of the body portion of the user by applying frequency filtering or movement pattern recognition.

18. The vibration therapy apparatus of claim 1, wherein the control unit comprises a mode selection switch to enable manual switching between a plurality of preset vibration modes.

19. The vibration therapy apparatus of claim 1, wherein the control unit is configured to gradually reduce the at least one vibration parameter after a therapy session.

20. The vibration therapy apparatus of claim 1, further comprising one or more photobiomodulation modules.
